(19) United States
(12) Patent Application Publication
(10) Pub. No.: US 2017/0219587 A1
(43) Pub. Date: Aug. 3, 2017

(54) METHODS FOR ASSESSING BIOLOGICAL SAMPLE QUALITY

(71) Applicant: INDIVUMED GMBH, Hamburg (DE)

(72) Inventors: Hartmut JUHL, Hamburg (DE); Kerstin DAVID, Hamburg (DE); Florian T. UNGER, Hamburg (DE)

(73) Assignee: INDIVUMED GMBH, Hamburg (DE)

(21) Appl. No.: 15/329,630

(22) PCT Filed: Jul. 30, 2015
(86) PCT No.: PCT/IB2015/001799

§ 371 Date: Jan. 27, 2017

Related U.S. Application Data
(60) Provisional application No. 62/031,247, filed on Jul. 31, 2014.

(51) Int. Cl. G01N 33/574; C12Q 1/68

(52) U.S. Cl. CPC G01N 33/57419; G01N 33/57438; C12Q 1/6886; G01N 2440/14; C12Q 2600/158; C12Q 2600/166

(57) ABSTRACT

Methods for assessing the quality of a tissue sample. In some aspects, methods are provided for determine the time that a biological sample has been exposed to cold ischemia condition by measuring the expression level of an mRNA and/or a phosphoprotein.

### CROSS-REFERENCE TO RELATED APPLICATIONS

This application claims the benefit of priority to U.S. Provisional Application Ser. No. 62/031,247, filed on Jul. 31, 2014, the entire contents of which are hereby incorporated by reference.

The invention was made with government support under Grant No. HHSN2612008000001E awarded by the National Institutes of Health. The government has certain rights in the invention.

### BACKGROUND OF THE INVENTION

1. Field of the Invention

The present invention relates generally to the fields of medicine, pathology and cell and molecular biology. More particularly, it concerns methods for clinical diagnostics, drug development and identification of predictive biomarkers.

2. Description of Related Art

The development of personalized medicine in oncology (determining an individual's disease risk, prognosis, and therapeutic options) is fostered by high-throughput analysis of molecular biomarkers in human cancer biospecimens (Zatloukal and Hainaut, 2010). Insufficient quality of such specimens may lead to spurious results and data misinterpretation (Vaught and Lockhart, 2012). Biospecimen quality depends on the pre-analytical conditions in which it was acquired (Juhl, 2010). Of critical importance is the time interval between reducing blood supply and removing the tissue (warm ischemia time), and the time interval between removing the tissue and preserving its molecular composition (cold ischemia time) (Huang et al., 2001; Spruessel et al., 2004; Espina et al., 2008; Hatzis et al., 2011; Gundisch et al., 2012). In addition, patients (cells) are exposed to drugs and/or are manipulated in ways that may influence expression profiles and pathway activity, resulting in inaccurate analytical data. However, there are no systematic studies analyzing the impact of pre-analytical factors. An understanding of tissue data variability in relation to processing techniques during and postsurgery would be desirable when testing surgical specimens for clinical diagnostics, drug development, or identification of predictive biomarkers.

### SUMMARY OF THE INVENTION

In one embodiment, provided herein is a method of determining a quality of a patient sample comprising (a) measuring (or selectively measuring) a level of HSP27 protein phosphorylation in the patient sample; and (b) determining the quality of the patient sample based on the HSP27 protein phosphorylation in the patient sample. For instance, in some aspects, a method comprises (a) measuring a level of expression of HSP27 protein and a level of phosphorylation of HSP27 protein (e.g., phosphorylation at Ser15) in the patient sample; and (b) determining the quality of the patient sample based on the percentage of HSP27 protein phosphorylation in the patient sample.

In a further embodiment, there is provided a method of determining a quality of a patient sample comprising (a) measuring (or selectively measuring) a level of phosphorylation of at least one, two, three, four or five proteins selected from the group consisting of HSP27, EGFR, AKT, mTOR, p70-S6K, GSK3-beta, MEK1/2, and ERK1/2 in the patient sample; and (b) determining the quality of the patient sample based on the phosphorylation of said proteins in the patient sample. For instance, a method can comprise (a) measuring a level of expression and a level of phosphorylation of at least one, two, three, four or five proteins selected from the group consisting of HSP27 (e.g., phosphorylation at Ser15), EGFR (e.g., phosphorylation at Tyr1173), AKT (e.g., phosphorylation at Ser473), mTOR (e.g., phosphorylation at Ser2448), p70-S6K (e.g., phosphorylation at Thr421 and/or Ser424), GSK3-beta (e.g., phosphorylation at Ser9), MEK1/2 (e.g., phosphorylation at Ser271 and/or Ser221), and ERK1/2 (e.g., phosphorylation at Thr202, Tyr204, Thr185 and/or Tyr187) in the patient sample; and (b) determining the quality of the patient sample based on the percentage of phosphorylation of said proteins in the patient sample. In various aspects, determining the quality of the patient sample may further comprise estimating the time period that the sample was exposed to cold ischemia conditions.

In some aspects, the method may further comprise measuring the level of mRNA expression of at least one gene in the sample. For example, a method may comprise measuring the level of mRNA expression from a gene selected from the group consisting of CYR61, RGS1, DUSP1, DUOX2, and SLC6A14. Thus, in some aspects, determining the quality of the patient sample is based on the percentage of phosphorylation of one or more proteins and on the expression levels of one or more mRNAs in the patient sample.

In some aspects, a change in the percentage of the protein in the patient sample that is phosphorylated as compared to a reference level may indicate the tissue quality. In certain aspects, the reference level may be the level of phosphorylation in a normal, non-diseased tissue, or in a sample from tissue having a known disease condition. In some cases a reference level it from a catalog or table. In further aspects, a reference level may be determined from a reference tissue. In some aspects, the reference tissue and sample tissue are obtained from the same patient (e.g., such as a tumor biopsy sample and an adjacent normal tissue sample).

In various aspects, the method may further comprise determining the percentage of at least a second, third or fourth protein in the patient sample that is phosphorylated. In certain aspects, the second, third or fourth protein may be selected from the group consisting of EGFR, AKT, mTOR, p70-S6K, GSK3-beta, MEK1/2, and ERK1/2. In certain aspects, the percentage of phosphorylation of Tyr1173 of EGFR, Ser 473 of AKT, Ser2448 of mTOR, Thr421 or Ser424 of p70-S6K, Ser9 of GSK3-beta, Ser271/221 of MEK1/2, or Thr202/Tyr204 or Thr185/Tyr187 of ERK1/2 may be determined. In various aspects, the method may further comprise determining the percentage of 5, 6, 7, 8, 9, or 10 proteins in the patient sample that are phosphorylated.

In a further embodiment of the invention, a method is provided for determining a quality of a patient sample comprising (a) measuring (or selectively measuring) a level of expression of at least one mRNA selected from the group consisting of CYR61, RGS1, DUSP1, DUOX2, and SLC6A14 in the patient sample; and (b) determining the quality of the patient sample based on the level of expression of the at least one mRNA in the patient sample. In certain aspects, the method may further comprise measuring the level of expression of a stable mRNA and determining a ratio of the at least one mRNA to the stable mRNA. In some aspects, a change in the ratio as compared to a reference level may indicate tissue quality. In some aspects, the stable mRNA may be EEF1A1. In various aspects, the method may further comprise measuring level of expression of 2, 3, 4, 5, 6, 7, 8, 9, or 10 mRNAs in the patient sample.

In further aspects, a method of the embodiments may further comprise determining the level (or percentage) of phosphorylation of at least one protein in the patient sample. For example, at least one protein may be selected from the groups consisting of HSP27 (e.g., phosphorylation at Ser15), EGFR (e.g., phosphorylation at Tyr1173), AKT (e.g., phosphorylation at Ser473), mTOR (e.g., phosphorylation at Ser2448), p70-S6K (e.g., phosphorylation at Thr421 and/or Ser424), GSK3-beta (e.g., phosphorylation at Ser9), MEK1/2 (e.g., phosphorylation at Ser271 and/or Ser221), and ERK1/2 (e.g., phosphorylation at Thr202, Tyr204, Thr185 and/or Tyr187). In certain aspects, the level of HSP27 phosphorylation is measured in additional to the expressions level of at least one mRNA. In further aspects, the assay may further comprise selectively measuring a level of expression and a level of phosphorylation of 5, 6, 7, 8, 9, or 10 proteins in the patient sample. Thus, in some aspects, determining the quality of the patient sample is based on the percentage of phosphorylation of one or more proteins and on the expression levels of one or more mRNAs in the patient sample.

In some aspects, the sample may be a tissue sample, such as a solid tissue sample (e.g., a biopsy sample). Samples for use according to the embodiments can be fresh (unfrozen or unfixed) samples, frozen samples, or a chemically fixed samples, such as, for example, a formalin-fixed, paraffin-embedded (FFPE) sample. In certain aspects, the tissue sample may be a section of tissue from a solid organ. In some aspects, the tissue sample is a colon, breast, kidney, liver, ovary, intestine, stomach, brain, lymph node, adrenal gland, thyroid, lung, esophageal, rectal, skin, prostate, cervical, or pancreas tissue sample. In further aspects, the tissue sample may be a tumor resection sample, such as, for example, a sample of a colorectal carcinoma or hepatic carcinoma.

In some aspects, the method may further comprise obtaining a sample from the patient (e.g., by directly sampling the patient). In other aspects, a sample is obtained from a third party, such as a hospital or healthcare worker.

In further aspects, a method comprises reporting the quality of a tissue sample analyzed in accordance with the embodiments (e.g., preparing a report estimating the time that the tissue sample was exposed to cold ischemia conditions). Reporting may comprise preparing an oral, written or electronic report. In some aspects, such a report is provided to the patient, a doctor, a hospital, or an insurance company.

Various aspects of the embodiments concern measuring mRNA and protein expression or protein phosphorylation. A wide range of techniques are known to those of skill in the art and may be used in such measurements. In certain aspects, the level of mRNA expression may be measured by quantitative real-time PCR, Northern blotting, in situ hybridization or an array hybridization. In some aspects, the level of expression or phosphorylation of a protein may be measured by ELISA, western blotting, mass spectrometry, a capillary immune-detection method, isoelectric focusing, an immune precipitation method or immunohistochemistry.

As used herein the phrase “selectively measuring” refers to methods wherein only a finite number of protein (e.g., phosphoprotein) or nucleic acid (e.g., mRNA) markers are measured rather than assaying essentially all proteins or nucleic acids in a sample. For example, in some aspects “selectively measuring” nucleic acid or protein markers can refer to measuring no more than 100, 75, 50, 25, 15, 10 or 5 different nucleic acid or protein (e.g., phosphoprotein) markers.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Flow chart of patient enrollment and tissue collection. Patients had (A) primary colorectal cancer and (B) metastasized colorectal cancer. Some patients with metastasized cancer had hepatic pedicle clamping (H.p.). Tissue preservation methods were: frozen in liquid nitrogen (FF) or formalin-fixed paraffin-embedded (FFPE).

FIG. 2. The variability of gene expression changes between patients, tissue type, surgery and tissue processing times. The figure shows the number of genes whose expression changed by more than 2-fold according to the tissue source and timing of pedicle clamping and postsurgical processing: pre, endoscopic biopsy presurgery (colon)/before hepatic pedicle clamping (liver); post, after clamping, 10′, 10 minutes after resection, 20′, 20 minutes after resection, and 45′, 45 minutes after resection. Bars represent mean numbers of gene expression changes.

FIG. 3A-3B. FIG. 3A, Hierarchical clustering of patients undergoing colon surgery and showing clustering in normal tissue (left) and primary tumors (right) across different timepoints. FIG. 3B, Hierarchical clustering of patients undergoing liver surgery and showing clustering in normal tissue (left) and metastatic tumors (right) across different timepoints.

FIG. 4A-4B. FIG. 4A, Protein expression in colon tissue. The figure shows changes of more than 2-fold in total protein expression of selected proteins, measured in normal colon and colorectal cancer tissue. Protein expression changes were compared: pre, endoscopic biopsy presurgery 10 minutes after resection; and 45′, 45 minutes after resection. FIG. 4B, Protein expression in liver tissue. The figure shows changes of more than 2-fold in total protein expression of selected proteins, measured in normal hepatic and metastatic tissue. Protein expression changes were compared: pre, before hepatic pedicle clamping; post, after clamping; 10′, 10 minutes after resection; 20′, 20 minutes after resection; and 45′, 45 minutes after resection.

FIGS. 5A-5D. FIGS. 5A-5B, Total protein expression (relative units) of p70-S6K, AKT, EGFR, ERK1/2, mTOR and GSK3β in normal and tumor colon tissue at four timepoints of tissue collection: pre, endoscopic biopsy presurgery; 10′, 10 minutes after resection; 20′, 20 minutes after resection; and 45′, 45 minutes after resection. *p<0.05; **p<0.01; ***p<0.001. Box plots indicate the 5%/95% confidence interval, median and standard deviation. FIGS. 5C-5D, Percentage of protein phosphorylation of p70-S6K, AKT, EGFR, ERK1/2, mTOR and GSK3β in normal and tumor colon tissue at four timepoints of tissue collection: pre, endoscopic biopsy presurgery; 10′, 10 minutes after resection; 20′, 20 minutes after resection; and 45′, 45 minutes after resection. *p<0.05; **p<0.01; ***p<0.001. Box plots indicate the 5%/95% confidence interval, median and standard deviation.

FIG. 6. Expression of EGFR in normal colon (left) and tumor tissue (right) in a subgroup of patients who showed at least 2-fold change in protein expression (up- or down-regulated) as determined by analyzing tissue lysates using a sandwich ELISA analysis provided by the MSD® technology.

FIG. 7. Representative immunohistochemistry for pAKT on formalin-fixed colon cancer tissue from one patient taken at four timepoints: (A) biopsy presurgery; (B) tissue fixed 10 minutes after resection; (C) tissue fixed 20 minutes after resection; and (D) tissue fixed 45 minutes after resection.

FIG. 8. Total protein expression (relative units) and percentage phosphorylation for HSP27 in (A) normal colon, (B) liver, (C) primary colon cancer, and (D) metastatic liver lesion tissue. Tissue was obtained: pre, endoscopic biopsy presurgery (colon)/before hepatic pedicle clamping; post, after clamping; 10′, 10 minutes after resection; 20′, 20 minutes after resection; and 45′, 45 minutes after resection. **p<0.01; ***p<0.001.

### DESCRIPTION OF ILLUSTRATIVE EMBODIMENTS

The present invention provides a correlation of the effects that warm and cold ischemia have on the molecular composition of a tissue specimen. As shown herein, specimens of normal and colorectal cancer (CRC) tissues removed during colon and liver resection surgery were obtained at the beginning of surgery and postsurgically, tissue was fixed at 10, 20, and 45 minutes. Specimens were analyzed from 50 patients with primary CRC and 43 with intrahepatic metastasis of CRC using a whole genome gene expression array. Additionally, protein expression and phosphorylation status in relation to tissue processing timepoints were quantified for proteins in the epidermal growth factor receptor pathway. Gene and protein expression data obtained from colorectal and liver specimens were determined to be influenced by tissue handling during surgery and by postsurgical processing/ischemia time. To obtain reliable expression data, tissue processing for research and diagnostic purposes needs to be highly standardized.

### I. THE PRESENT INVENTION

Pharmaceutical companies put significant efforts into the development of specific pathway inhibitors, and new drugs can emerge in high numbers from preclinical development programs. Identification of drug targets as stratification and predictive biomarkers in patient populations has become an important field in drug development; indeed, such biomarkers are thought to be essential for current and future patient care and an important strategy in controlling reimbursement costs in cancer care.

While DNA aberrations, such as activating mutations in specific genes, allow the identification to some extent of drug targets under various circumstances, this approach is of limited predictive value. Utilization of protein targets and cancer pathway activity as determined by quantifying phosphorylation of regulatory proteins can provide a much deeper insight into a patient's individual tumor biology. However, a major challenge for the discovery and development of predictive drug targets is the need for tissue samples that truly represent the reality of a patient's tumor biology. As most tissues are surgical specimens, a major concern is the risk of modified expression levels because of tissue manipulation during and after surgery. Understanding the effects of surgical manipulation on cancer biomarkers will be an important part of the knowledge base upon which such biomarkers can be fully utilized to benefit patients.

Stemming the blood supply rapidly induces hypoxia and cellular stress and is thought to affect the molecular composition of cells and thus, the analytical data derived from these tissues. In addition, during surgery, patients (and, naturally, their cells) are exposed to various kinds of drugs given by the anesthetist.

In the studies provided herein, it was demonstrated that human tissue is highly susceptible to surgical factors and prolonged postsurgical ischemia, and standardized handling of tissue is an important factor for subsequent analysis. Analyzing stress and ischemia markers such as HSP27 and HIF1A, it was found that in normal liver tissue, HSP27 phosphorylation was significantly increased at 10 minutes after hepatic pedicle clamping and further increased throughout post-resection ischemia in a time-dependent manner. In normal colon tissue, HSP27 phosphorylation increased in an almost linear time-dependent manner between 10′ and 45′ postsurgery. Hence, HSP27 phosphorylation appears to be a marker for pre- and post-resection tissue quality. A significant increase in HIF1A phosphorylation levels at 10′ postsurgery of colorectal tumors was also found, using a sandwich ELISA detection technique (i.e., MSD® technology); however, these levels declined again at later timepoints.

In individual patients the expression of more than 4,000 genes were altered and up to 60% of patients with primary CRC showed more than a 2-fold expression change in proteins and their phosphorylation. In general, the impact on the molecular composition was more severe in tumor tissue compared to normal tissue, likely due to the higher activity of tumor cells compared to normal cells. The most striking changes were observed during warm ischemia and early cold ischemia (10′ postsurgery); gene and protein expression changes during prolonged cold ischemia were surprisingly less prominent though still significant. Interestingly, up to 690 (mean 118) genes were already affected in individual patients by just clamping the hepatic pedicle for 10 minutes.

With regard to gene and protein expression data, inter-individual variability was observed across patients. Most of these patients, however, appeared to follow a similar pattern of gene expression changes. By only using those patients for further gene expression analyses, significant up-regulation of several transcription factors and signaling molecules of the extracellular matrix such as cysteine-rich angiogenic inducer 61 (CYR61, CCN1) and the regulator of G-protein signaling 1 (RGS1) was identified. CYR61 is a matrix cell-adhesion molecule. Depending on the context, it promotes cell proliferation, survival, apoptosis, or angiogenesis by binding to distinct integrins and plays an important role in wound repair (Lau et al., 2011). RGS1 attenuates the signaling activity of G-proteins by fostering GTP hydrolysis and has various immunomodulatory functions (Bansal et al., 2007). Simultaneously, the expression of several genes from colonic enterocytes was down-regulated.

On the protein level, the variability between patients was also high. Protein levels and their phosphorylation status showed increasing and decreasing levels between different patients and timepoints. However, the most consistent molecular change analyzed during tissue resection and post-surgical ischemia was a decrease or lower level in protein phosphorylation, observed for AKT, mTOR, p70-S6K, GSK3-beta, and ERK1/2 from colon tissue. Dephosphorylation of ERK1/2 is particularly interesting with regard to the fact that an up-regulated gene expression was found for dual specificity phosphatase 1 (DUSP1, MKP1). This protein can dephosphorylate MAPK (ERK) in the cell nucleus and thus attenuates MAPK signaling (Lawan et al., 2013).

The decline in phosphorylation status was also observed by immunohistochemical staining for pERK, pAKT and pEGFR in colon tissue, where staining intensity was often lower in postsurgery compared to presurgery samples. This has important consequences, since immunohistochemistry is frequently used to determine activation of signaling pathways in individual cancer specimens in order to determine personalized treatment options. The pathologist analyzing post-resection specimens needs to be aware of the alterations that can be induced by cold ischemia time and therefore needs precise information about the conditions under which the tissue was procured. Since DUSP1, CYR61, and RGS1 gene expression was up-regulated in both normal and CRC tissue upon tissue resection, these genes may represent interesting candidates for biomarkers of post-resection tissue quality.

The present studies also identified candidate “housekeeping” or relatively stable genes, the expression of which was not altered by tissue resection and post-surgical cold ischemia. In this regard, the EEF1A1 gene appears to be particularly interesting. Its CV was very low across all four timepoints, both in normal colon tissue and in CRC tissue. EEF1A1 is known to be constitutively expressed in many tissues and under various conditions and has been described as a useful housekeeping gene for gene expression analyses (Maltseva et al., 2013). Further candidates for reference genes have been identified. Interestingly, genes that are frequently used as reference genes such as beta2-microglobulin or beta2-tubulin did not show constitutive expression throughout resection and post-surgical ischemia and therefore do not appear suitable as housekeeping genes under the described conditions.

The data presented herein are largely in agreement with a recent report that described fluctuations of protein levels and protein phosphorylation in human intestine tissue samples as a consequence of different ischemic conditions before preservation (Gundisch et al., 2012). The authors reported that a general trend towards up- or down-regulation of proteins was not evident due to pronounced inter-individual variability. Due to the larger number of samples that were investigated in the present study, a general trend towards up- and down-regulation of proteins and their phosphorylation status was demonstrated despite the occurrence of high inter-individual variability.

In summary, the present data show a significant difference in the molecular composition of tissue specimens collected after tumor resection compared to specimens collected via colonoscopy before tumor resection. This difference is larger than the difference between various post-resection times between 10 and 45 minutes. The observed effect is either due to warm and cold ischemia, and/or the anesthesia/surgical procedure itself, and the manipulation of the tissue. In general, kinase proteins become dephosphorylated, which may result in decreased intensity of immunohistochemical staining. The ratio between phosphorylated and total HSP27 protein has emerged as a marker for tissue quality, since it demonstrates an almost linear increase with prolonged cold ischemia time in all analyzed tissue types.

This study presents an important contribution to the understanding of molecular changes that are being introduced into tissue samples during the pre-analytical phase, i.e. by the tissue collection procedure itself and the surgical procedures prior tissue collection. For the first time, a full list of genes is provided whose expression is altered (with a ≧2-fold change) due to tissue processing and surgical manipulation. In addition, the analysis of regulatory pathway proteins and specific growth factor receptors, such as EGFR, requires the use of highly standardized and rapid tissue processing techniques. Once standardized techniques have been validated, analysis of the expression of regulatory pathway proteins may be valuable as predictive markers of targeted therapies.

### II. BIOMARKER DETECTION

The expression of biomarkers such as but not limited to gene and/or protein expression (e.g., expression of mRNAs or phosphoproteins) may be measured by a variety of techniques that are well known in the art. Quantifying the levels of the messenger RNA (mRNA) of a biomarker may be used to measure the expression of the biomarker. Alternatively, quantifying the levels of the protein product of a biomarker may be used to measure the expression of the biomarker. Additional information regarding the methods discussed below may be found in Ausubel et al. (2003) or Sambrook et al. (1989). One skilled in the art will know which parameters may be manipulated to optimize detection of the mRNA or protein of interest.

In some embodiments, said obtaining expression information may comprise RNA quantification, e.g., cDNA microarray, quantitative RT-PCR, in situ hybridization, Northern blotting or nuclease protection. Said obtaining expression information may comprise protein quantification and/or quantitation of protein post-translational modifications such as phosphorylation. In some cases, such quantification comprises performing immunohistochemistry, an ELISA (e.g., a sandwich ELISA, such by use of MSD® technology), a radioimmunoassay (RIA), an immunoradiometric assay, a fluoroimmunoassay, a chemiluminescent assay, a bioluminescent assay, a gel electrophoresis, a Western blot analysis, a mass spectrometry analysis, a protein microarray, a capillary protein immune detection system, such the NanoPro1000™ or related technologies.

In some aspects, a marker level (scuh as phosphpoprtein or mRNA level) may be compared to the level of a control marker or with the corresponding marker from a control, sample. For example, in some cases the control maker is a biomarker (e.g., a protein, phosphoprotein or mRNA) that displays consistent stable levels duint ischemia exposure. Likewise, in some aspects a control sample is a sample that has not be exposed to ischemia or that has been isechemic for a known time period.

Control marker levels or marker levels from a control sample may be determined at the same time as a test sample (e.g., in the same experiment) or may be a stored value or set of values, e.g., stored on a computer, or on computer-readable media. If the latter is used, new sample data for the selected marker(s), obtained from initial or follow-up samples, can be compared to the stored data for the same marker(s) without the need for additional control experiments.

A. Methods of Protein Detection

In some aspects, measuring the expression of said genes comprises measuring protein expression levels. Measuring protein expression levels may comprise, for example, performing an ELISA, Western blot, immunohistochemistry, or binding to an antibody array. In certain aspects, determining a level of a phosphoprotein in a sample comprises contacting the sample with a phosphorylation specific antibody to the indicated phosphoprotein.

Immunohistochemical staining may also be used to measure the differential expression of a plurality of biomarkers. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art. Single- and Multi-probe kits are available from commercial suppliers, e.g., Meso Scale Discovery (MSD). These kits include the kits referenced in the Examples hereto.

An antibody microarray may also be used to measure the differential expression (and/or differential phosphoylation) of a plurality of biomarkers. For this, a plurality of antibodies is arrayed and covalently attached to the surface of the microarray or biochip. A protein extract containing the biomarker proteins of interest is generally labeled with a fluorescent dye or biotin. The labeled biomarker proteins are incubated with the antibody microarray. After washes to remove the unbound proteins, the microarray is scanned. The raw fluorescent intensity data may be converted into expression values using means known in the art.

B. Methods of Nucleic Acid Detection

In another aspect, measuring expression of said genes comprises measuring RNA expression levels. Measuring RNA expression levels may comprise performing RT-PCR, Northern blot, in situ hybridization or an array hybridization. Preferably, measuring the expression level of the genes comprises performing RT-PCR (e.g., real time RT-PCR).

A nucleic acid microarray may be used to quantify the differential expression of a plurality of biomarkers. Microarray analysis may be performed using commercially available equipment, following manufacturer's protocols, such as by using the Affymetrix GeneChip® technology (Santa Clara, Calif.) or the Microarray System from Incyte (Fremont, Calif.). For example, single-stranded nucleic acids (e.g., cDNAs or oligonucleotides) may be plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with the robust multichip average (RMA) algorithm to generate expression values.

Quantitative real-time PCR (qRT-PCR) may also be used to measure the differential expression of a plurality of biomarkers. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

A non-limiting example of a fluorescent reporter probe is a TaqMan® probe (Applied Biosystems, Foster City, Calif.). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR may be performed using a reference standard. The ideal reference standard is expressed at a constant level among different tissues, and is unaffected by the experimental treatment. Suitable reference standards include, but are not limited to, mRNAs for the housekeeping genes glyceraldehyde-3-phosphate-dehydrogenase (GAPDH) and β-actin. The level of mRNA in the original sample or the fold change in expression of each biomarker may be determined using calculations well known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker mRNA or protein, respectively). The target, in turn, is also tagged with a fluorescent reporter. Hence, there are two sources of color, one from the bead and the other from the reporter molecule on the target. The beads are then incubated with the sample containing the targets, of which up to 100 may be detected in one well. The small size/surface area of the beads and the three dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

### III. DEFINITIONS

As used herein, the term “biological sample” is used in its broadest sense and can refer to a bodily sample obtained from a subject (e.g., a human). For example, the biological sample can include a “clinical sample”, i.e., a sample derived from a subject. Such samples can include, but are not limited to: peripheral bodily fluids, which may or may not contain cells, e.g., blood, urine, plasma, mucous, bile pancreatic juice, supernatant fluid, and serum; tissue or fine needle biopsy samples; and archival samples with known diagnosis, treatment and/or outcome history. Biological samples may also include sections of tissues, such as frozen sections taken for histological purposes. The term “biological sample” can also encompass any material derived by processing the sample. Derived materials can include, but are not limited to, cells (or their progeny) isolated from the biological sample and proteins extracted from the sample. Processing of the biological sample may involve one or more of, filtration, distillation, extraction, concentration, fixation, inactivation of interfering components, addition of reagents, and the like.

By “subject” or “patient” is meant any single subject for which therapy or diagnostic test is desired. In this case the subjects or patients generally refer to humans. Also intended to be included as a subject are any subjects involved in clinical research trials not showing any clinical sign of disease, or subjects involved in epidemiological studies, or subjects used as controls.

As used herein, “increased expression” refers to an elevated or increased level of expression of a marker (e.g., mRNA, protein or phosphoprotein expression) in a sample (e.g., relative to a suitable control marker, control sample or time point), wherein the elevation or increase in the level of gene expression is statistically significant (p<0.05). Whether an increase in the expression of a marker in a sample relative to a control is statistically significant can be determined using an appropriate t-test (e.g., one-sample t-test, two-sample t-test, Welch's t-test) or other statistical test known to those of skill in the art.

As used herein, “decreased expression” refers to a reduced or decreased level of expression of a marker (e.g., mRNA, protein or phosphoprotein expression) in a sample (e.g., relative to a suitable control marker, control sample or time point), wherein the reduction or decrease in the level of gene expression is statistically significant (p<0.05). In some embodiments, the reduced or decreased level of marker expression can be a complete absence of expression. Whether a decrease in the expression of a marker in a sample relative to a control is statistically significant can be determined using an appropriate t-test (e.g., one-sample t-test, two-sample t-test, Welch's t-test) or other statistical test known to those of skill in the art.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Sample Collection and Preparation

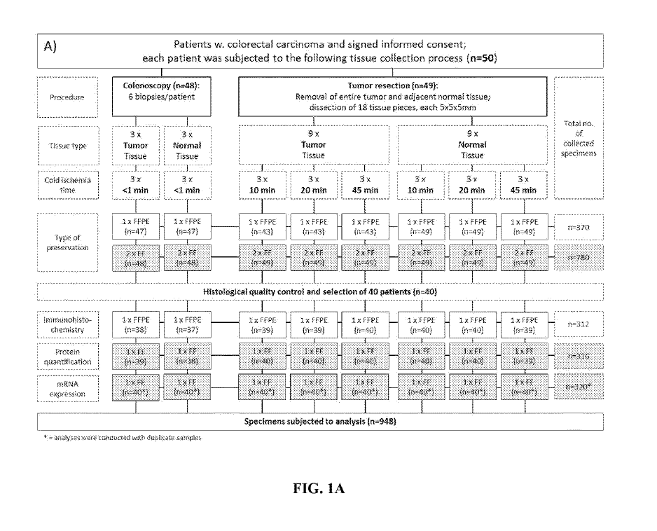

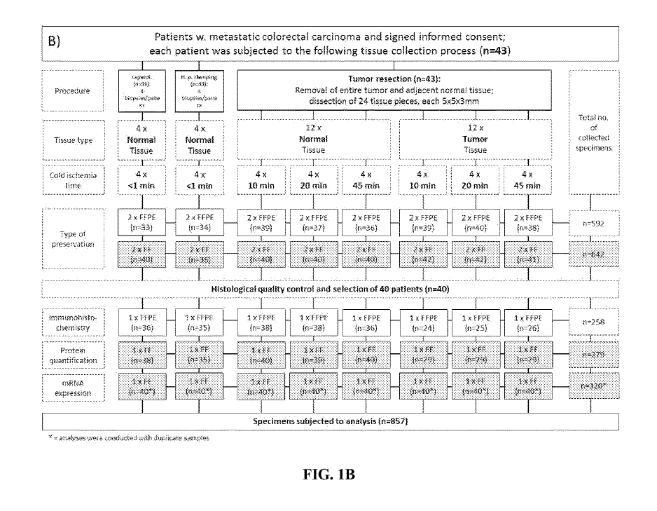

Fifty patients with primary colorectal cancer (CRC) and 43 with intrahepatic metastasis of CRC were enrolled in the study. From the 50 patients with a primary tumor, 370 formalin-fixed paraffin-embedded (FFPE) and 780 frozen in liquid nitrogen (FF) tissue samples were collected, and from the 43 patients with metastasized cancer, 592 FFPE and 642 FF tissue samples were collected. All samples were subjected to morphological quality control (FIGS. 1A-1B).

All patients who were scheduled for tumor resection surgery gave informed consent to be enrolled in the study. Only patients with a tumor larger than 3 cm in diameter were enrolled. Patients who had received chemotherapy or radiation therapy <3 weeks before surgery were excluded. Specially trained study nurses were present during all surgeries. They performed tissue processing in the surgical unit and clinical data documentation to assure that the same standardized operating procedures have been applied to all patients. The study was conducted at three sites in Hamburg, Germany, and received approval by the competent ethics review committee of the medical association Hamburg under reference No. PV3342.

After induction of anesthesia, patients with primary CRC underwent a colonoscopy, upon which three biopsies were taken from the tumor and three biopsies from the adjacent normal tissue (presurgery) (FIGS. 1A-1B). For patients with hepatic metastasis of CRC, four pieces of tissue were taken from normal liver parenchyma before the start of liver resection, i.e. just before clamping of the hepatic artery (presurgery). About 10 minutes after hepatic pedicle clamping (post-clamping), another four tissue samples were collected from the normal liver parenchyma (postsurgery).

For all patients, 12 tissue samples were each collected from the tumor tissue and the adjacent normal tissue after resection of the tumor and adjacent normal tissue. These 24 samples were divided into three groups, each exposed to a cold ischemia time of 10 minutes (10′), 20 minutes (20′), and 45 minutes (45′), respectively. Every tissue sample had an approximate size of 5×5×5 mm and an approximate weight of 120 mg. For each timepoint and tissue type (normal or tumor), half of the tissues were immediately stored in the vapor phase of liquid nitrogen, while the other half was immersion fixed in 4% buffered formaldehyde (FIGS. 1A-1B).

All tissue specimens in formaldehyde were immersion fixed for 16 to 72 hours. Thereafter, they were weighed and placed in 70% ethanol for a maximum of 24 hours until further processing. Processing was conducted with an automated system (Microm tissue processor STP 420 D Thermo Scientific, Dreieich, Germany) resulting in the embedding of tissues in paraffin (Paraplast).

From each formalin-fixed paraffin-embedded (FFPE) and frozen in liquid nitrogen (FF) tissue specimen, one section was stained with hematoxylin-eosin and evaluated under a light microscope in order to verify the presence of tumor and normal tissue, respectively. Tumor content was 10%-90% in tumor samples and 0% in all adjacent normal samples.

After histological quality control, FFPE and FF samples were selected for the following molecular analyses: i) quantification of total and phosphorylated protein by a medium-throughput enzyme-linked immunosorbent assay technology (Meso Scale Discovery [MSD]), ii) semi-quantitative evaluation of protein expression by immunohistochemistry, and iii) gene expression profiling on total RNA extracts using an Affymetrix whole genome chip (FIGS. 1A-1B).

### Example 2—Quantification of Proteins

Forty FF specimens were used. Tissue lysates were prepared by cutting and homogenizing a 20 m slice from each FF specimen. The resulting tissue lysate was subjected to a bicinchoninic acid protein assay (BCA kit; Sigma, Steinheim, Germany) to determine protein concentration. Quantification of proteins was conducted using 96-well plates with capture antibodies based on the assay platform from MSD (Gaithersburg, Md., USA). The following assay kits were used: HIF1alpha singleplex, HSP27/pHSP27(Ser15) duplex, EGFR/pEGFR(Tyr1173) duplex, AKT/pAKT(Ser473) duplex, mTOR/pmTOR(Ser2448) duplex, p70-S6K/pp70-S6K(Thr421, Ser424) duplex, GSK3-beta/pGSK3-beta(Ser9) duplex, MEK1/2/pMEK1/2(Ser217/221) duplex and ERK1/2/pERK1/2(Thr202/Tyr204, Thr185/Tyr187) duplex.

Assays were performed using 10 g of tissue lysate according to the manufacturers' instructions and analyzed with the SECTOR Imager platform (MSD). Analyses were conducted in triplicate and arithmetic mean values were calculated. Mean values of post-surgery samples were compared against presurgery samples from the same patient. The percentage of phosphorylation was calculated according to the formula: phosphorylation (%)=(DFx phosphorylated protein)/(phosphorylated protein+total protein)×100 with distribution factor (DF)=2. If the phosphorylation (%) was >100, DF was adjusted. As run controls, lysates of stimulated human cells were produced and employed as positive and negative controls.

### Example 3—Immunohistochemistry

5 μm sections from FFPE tissue were mounted on glass slides, air dried at 56° C. overnight and subjected to immunostaining using an automated platform (Ventana Discovery XT, Tucson, Ariz., USA). The following primary antibodies and dilutions were used: pERK1/2 1:300, pAkt(Ser473) 1:30, pEGFR 1:110, pmTOR 1:130, pHer3 1:70 (all from Cell Signaling Technology Inc., Danvers, Mass., USA). After staining, sections were treated with ascending ethanol concentrations and xylene and were finally covered with Pertex (Medite GmbH, Burgdorf, Germany). Sections were examined under a light microscope by a pathologist. Tumor cell staining was classified as absent, weak, moderate or strong and a staining score was calculated based on the extent of staining according to the formula: score=3× percentage of strongly stained tumor cells +2× percentage of moderately stained tumor cells +1× percentage of weakly stained tumor cells.

### Example 4—The Effect of Tissue Processing Timepoint on Gene Expression

For gene expression analysis, total RNA was extracted in duplicates from every frozen tissue block. Briefly, tissues were homogenized and RNA was isolated in two steps using phenol chloroform extraction and the RNeasy MinElute Cleanup Kit (Qiagen, Hilden, Germany) according to the manufacturer's instructions. RNA quality was evaluated based on 18S and 28S ribosomal RNA peaks using the Agilent 2100 bioanalyzer (Agilent Technologies, Berlin, Germany). Only RNA samples with an RNA integrity number ≧7 were used for gene expression analysis. RNA samples were analyzed in replicates using oligonucleotide microarrays (GeneChip Human Genome U133 Plus 2.0) based on the Affymetrix GeneChip™ technology (Affymetrix Inc., Santa Clara, Calif., USA).

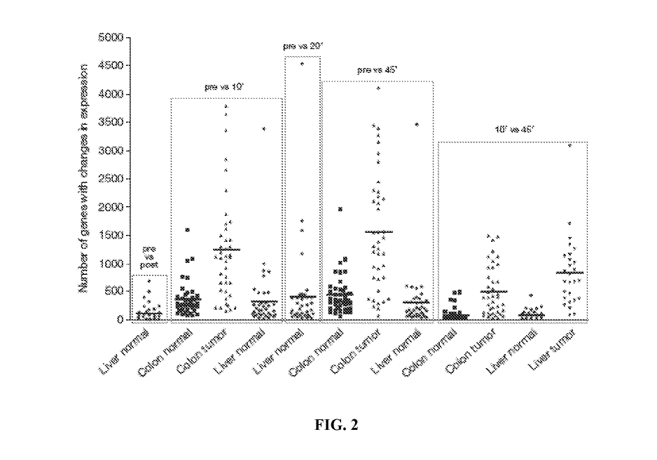

Ten-minute clamping time of the hepatic artery significantly changed the expression of up to 690 (mean 118) genes in normal liver. The number of affected genes increased with surgery time (FIG. 2). While some genes normalized (vs. first biopsy), other genes has significant changes in expression level with prolonged surgery and postsurgical tissue processing time. The number of affected genes in normal liver and normal colon was similar (see Table 1 below).

TABLE 1
Differentially expressed genes (≧2-fold) in normal colon and liver tissues.
Expression recorded: pre, before hepatic pedicle clamping; post, after
clamping, 10′, 10′ after resection; and 45′, 45′ after resection.

| | | | Pre | Pre |
|---|---|---|---|---|
| | | | vs. | vs. |
| Probe ID | Gene | Protein | 10′ | 45′ |
| 1555827_at | CCNL1 | Cyclin L1 | * | * |
| 201324_at | EMP1 | Epithelial membrane protein 1 | * | * |
| 201325_s_at | | | * | |
| 202499_s_at | SLC2A3 | Solute carrier family 2 (facilitated | * | * |
| | | glucose transporter), member 3 | | |
| 202672_s_at | ATF3 | Activating transcription factor 3 | * | |
| 202988_s_at | RGS1 | Regulator of G-protein signaling 1 | * | * |
| 216834_at | | | * | * |
| 215034_s_at | TM4SF1 | Transmembrane 4 L six family member | * | * |
| | | 1 | | |
| 227697_at | SOCS3 | Suppressor of cytokine signaling 3 | * | * |
| 232304_at | PELI1 | Pellino homolog 1 (Drosophila) | * | * |
| 36711_at | MAFF | V-maf musculoaponeurotic fibrosarcoma | * | |
| | | oncogene homolog F (avian) | | |
| 202291_s_at | MGP | Matrix GLA protein | | * |
| 209101_at | CTGF | Connective tissue growth factor | | * |
| 228335_at | CLDN11 | Claudin 11 | | * |
| 202859_x_at | IL8 | Interleukin 8 | | * |
| 228528_at | LOC100286909 | Hypothetical protein LOC100286909 | | * |

* p < 0.05.

In contrast to normal tissue, the variability of gene expression in relation to surgery and postsurgical processing time was significantly higher in cancer tissue. Within 10′ and 45′ postsurgery, up to 3,087 (mean 830) genes in metastatic liver CRC tumors showed ≧2-fold and significant difference in expression. In primary CRC tissue, comparison between presurgery and 10′ postsurgery biopsies identified up to 3,792 (mean 1,234) genes and 45′ postsurgery biopsies identified up to 4,116 (mean 1,553) genes. Tables 2-5 (below) summarize all genes that showed a significant and ≧2-fold change in expression during surgery and postsurgical processing.

TABLE 2
Differentially expressed genes in normal colon tissue according to when
tissue was obtained. P-values represent the comparison between tissue obtained
presurgery and 10 minutes after resection; the 70 regulated genes with ≧2-fold
(statistically significant) change in expression are listed.

| | | | | Proportional |
|---|---|---|---|---|
| Probe ID | Gene | Protein | P-value | change |
| 1555847_a_at | LOC284454 | Hypothetical LOC284454 | 9.54667E−12 | 2.50 |
| 1555938_x_at | VIM | Vimentin | 3.70146E−11 | 2.65 |
| 1557285_at | AREGB | Amphiregulin B | 4.38611E−07 | 2.53 |
| 1564796_at | EMP1 | Epithelial membrane protein | 1.96927E−10 | 2.84 |
| | | 1 | | |
| 201041_s_at | DUSP1 | Dual specificity phosphatase | 1.20966E−17 | 3.60 |
| | | 1 | | |
| 201289_at | CYR61 | Cysteine-rich, angiogenic | 1.56579E−18 | 5.62 |
| | | inducer, 61 | | |
| 201631_s_at | IER3 | Immediate early response 3 | 1.52502E−06 | 2.02 |
| 201693_s_at | EGR1 | Early growth response 1 | 5.45124E−09 | 2.39 |
| 201694_s_at | EGR1 | Early growth response 1 | 1.92369E−12 | 4.06 |
| 201739_at | SGK1 | Serum/glucocorticoid | 4.02631E−14 | 4.27 |
| | | regulated kinase 1 | | |
| 201893_x_at | DCN | Decorin | 4.24098E−10 | 2.07 |
| 202291_s_at | MGP | Matrix Gla protein | 3.32429E−08 | 3.16 |
| 202672_s_at | ATF3 | Activating transcription | 1.29365E−12 | 3.82 |
| | | factor 3 | | |
| 202988_s_at | RGS1 | Regulator of G-protein | 4.20768E−21 | 5.07 |
| | | signaling 1 | | |
| 203821_at | HBEGF | Heparin-binding EGF-like | 1.92717E−10 | 3.35 |
| | | growth factor | | |
| 204622_x_at | NR4A2 | Nuclear receptor subfamily | 1.66002E−09 | 3.82 |
| | | 4, group A, member 2 | | |
| 205200_at | CLEC3B | C-type lectin domain family | 1.65903E−11 | 2.38 |
| | | 3, member B | | |
| 205239_at | AREG | Amphiregulin | 9.33122E−10 | 3.01 |
| 205382_s_at | CFD | Complement factor D | 1.38175E−09 | 2.14 |
| | | (adipsin) | | |
| 205440_s_at | NPY1R | Neuropeptide Y receptor Y1 | 6.18072E−10 | 2.17 |
| 206577_at | VIP | Vasoactive intestinal peptide | 1.27434E−05 | 2.41 |
| 208078_s_at | SIK1 | Salt-inducible kinase 1 | 8.60542E−09 | 3.15 |
| 208763_s_at | TSC22D3 | TSC22 domain family, | 1.07833E−11 | 2.33 |
| | | member 3 | | |
| 209101_at | CTGF | Connective tissue growth | 7.37383E−16 | 3.06 |
| | | factor | | |
| 209189_at | FOS | FBJ murine osteosarcoma | 1.26409E−18 | 6.81 |
| | | viral oncogene homolog | | |
| 209335_at | DCN | Decorin | 1.20046E−10 | 2.34 |
| 210764_s_at | CYR61 | Cysteine-rich, angiogenic | 5.62682E−15 | 3.56 |
| | | inducer, 61 | | |
| 211896_s_at | DCN | Decorin | 1.3672E−09 | 2.33 |
| 212225_at | EIF1 | Eukaryotic translation | 3.33258E−14 | 2.06 |
| | | initiation factor 1 | | |
| 212977_at | CXCR7 | Chemokine (C-X-C motif) | 4.73779E−10 | 2.05 |
| | | receptor 7 | | |
| 213068_at | DPT | Dermatopontin | 7.0909E−10 | 2.52 |
| 214038_at | CCL8 | Chemokine (C-C motif) | 5.52415E−14 | 2.84 |
| | | ligand 8 | | |
| 216248_s_at | NR4A2 | Nuclear receptor subfamily | 8.88079E−09 | 3.80 |
| | | 4, group A, member 2 | | |
| 216834_at | RGS1 | Regulator of G-protein | 1.65149E−22 | 5.04 |
| | | signaling 1 | | |
| 218541_s_at | C8orf4 | Chromosome 8 open reading | 2.39257E−12 | 4.00 |
| | | frame 4 | | |
| 218730_s_at | OGN | Osteoglycin | 1.11377E−08 | 2.36 |
| 219087_at | ASPN | Asporin | 1.46805E−10 | 2.03 |
| 219230_at | TMEM100 | Transmembrane protein 100 | 2.2327E−14 | 2.15 |
| 219295_s_at | PCOLCE2 | Procollagen C-endopeptidase | 3.16146E−08 | 2.13 |
| | | enhancer 2 | | |
| 220468_at | ARL14 | ADP-ribosylation factor-like | 7.67767E−11 | 2.10 |
| | | 14 | | |
| 222162_s_at | ADAMTS1 | ADAM metallopeptidase | 4.62579E−11 | 2.54 |
| | | with thrombospondin type 1 | | |
| | | motif, 1 | | |
| 222722_at | OGN | Osteoglycin | 1.34695E−08 | 2.79 |
| 223121_s_at | SFRP2 | Secreted frizzled-related | 1.04636E−08 | 2.18 |
| | | protein 2 | | |
| 223122_s_at | SFRP2 | Secreted frizzled-related | 1.07874E−09 | 3.21 |
| | | protein 2 | | |
| 224657_at | ERRFI1 | ERBB receptor feedback | 7.42432E−10 | 2.09 |
| | | inhibitor 1 | | |
| 227099_s_at | C11orf96 | Chromosome 11 open | 7.20862E−12 | 3.09 |
| | | reading frame 96 | | |
| 227404_s_at | EGR1 | Early growth response 1 | 1.39748E−10 | 3.94 |
| 227697_at | SOCS3 | Suppressor of cytokine | 0.000387697 | 2.15 |
| | | signaling 3 | | |
| 228335_at | CLDN11 | Claudin 11 | 1.03247E−10 | 2.32 |
| 230494_at | — | — | 2.71373E−11 | 2.40 |
| 243509_at | — | — | 5.3854E−16 | 2.13 |
| 36711_at | MAFF | V-maf musculoaponeurotic | 1.6224E−09 | 2.66 |
| | | fibrosarcoma oncogene | | |
| | | homolog F (avian) | | |
| 38037_at | HBEGF | Heparin-binding EGF-like | 2.60564E−09 | 2.50 |
| | | growth factor | | |
| 1554436_a_at | REG4 | Regenerating islet-derived | 0.000125463 | −2.88 |
| | | family, member 4 | | |
| 203649_s_at | PLA2G2A | Phospholipase A2, group IIA | 5.92788E−06 | −2.42 |
| | | (platelets, synovial fluid) | | |
| 205844_at | VNN1 | Vanin 1 | 0.000102012 | −3.07 |
| 207214_at | SPINK4 | Serine peptidase inhibitor, | 0.000340689 | −2.75 |
| | | Kazal type 4 | | |
| 207397_s_at | HOXD13 | Homeobox D13 | 9.4269E−08 | −2.95 |
| 212531_at | LCN2 | Lipocalin 2 | 1.15271E−06 | −2.68 |
| 212768_s_at | OLFM4 | Olfactomedin 4 | 0.003466089 | −2.27 |
| 214604_at | HOXD11 | Homeobox D11 | 4.34958E−09 | −2.12 |
| 219727_at | DUOX2 | Dual oxidase 2 | 3.35786E−08 | −3.69 |
| 219795_at | SLC6A14 | Solute carrier family 6 | 1.0087E−05 | −3.66 |
| | | (amino acid transporter), | | |
| | | member 14 | | |
| 221091_at | INSL5 | Insulin-like 5 | 5.07532E−05 | −2.69 |
| 223447_at | REG4 | Regenerating islet-derived | 0.000132695 | −2.50 |
| | | family, member 4 | | |
| 228592_at | MS4A1 | Membrane-spanning 4- | 0.000474385 | −2.21 |
| | | domains, subfamily A, | | |
| | | member 1 | | |
| 229152_at | C4orf7 | Chromosome 4 open reading | 0.001397357 | −2.49 |
| | | frame 7 | | |
| 229400_at | HOXD10 | Homeobox D10 | 8.95393E−08 | −2.89 |
| 236681_at | HOXD13 | Homeobox D13 | 5.02826E−08 | −2.05 |
| 238847_at | — | — | 4.27233E−08 | −2.94 |

TABLE 3
Differentially expressed genes in normal colon tissue according to a comparison
of timepoint. P-values represent the comparison between tissue obtained
presurgery and 45 minutes after resection; the 96 regulated genes with ≧2-fold
(statistically significant) change in expression are listed.

| | | | | Proportional |
|---|---|---|---|---|
| Probe ID | Gene | Protein | P-value | change |
| 1552296_at | BEST4 | Bestrophin 4 | 1.28199E−06 | 2.09 |
| 1555847_a_at | LOC284454 | Hypothetical LOC284454 | 1.49773E−14 | 2.17 |
| 1555938_x_at | VIM | Vimentin | 1.98675E−18 | 2.53 |
| 1557285_at | AREGB | Amphiregulin B | 6.55772E−09 | 2.35 |
| 1564796_at | EMP1 | Epithelial membrane protein | 7.68089E−17 | 3.82 |
| | | 1 | | |
| 201041_s_at | DUSP1 | Dual specificity phosphatase | 2.99931E−23 | 4.45 |
| | | 1 | | |
| 201289_at | CYR61 | Cysteine-rich, angiogenic | 2.83776E−22 | 5.86 |
| | | inducer, 61 | | |
| 201324_at | EMP1 | Epithelial membrane protein | 9.86918E−12 | 2.09 |
| | | 1 | | |
| 201325_s_at | EMP1 | Epithelial membrane protein | 1.90042E−11 | 2.09 |
| | | 1 | | |
| 201465_s_at | JUN | Jun proto-oncogene | 6.16667E−17 | 2.02 |
| 201466_s_at | JUN | Jun proto-oncogene | 4.02899E−21 | 2.43 |
| 201631_s_at | IER3 | Immediate early response 3 | 1.04871E−08 | 2.03 |
| 201693_s_at | EGR1 | Early growth response 1 | 1.42355E−12 | 2.60 |
| 201694_s_at | EGR1 | Early growth response 1 | 1.79272E−18 | 4.78 |
| 201739_at | SGK1 | Serum/glucocorticoid | 2.74262E−16 | 4.62 |
| | | regulated kinase 1 | | |
| 201893_x_at | DCN | Decorin | 2.54325E−11 | 2.21 |
| 202068_s_at | LDLR | Low density lipoprotein | 1.13748E−08 | 2.06 |
| | | receptor | | |
| 202241_at | TRIB1 | Tribbles homolog 1 | 1.49548E−14 | 2.00 |
| | | (Drosophila) | | |
| 202291_s_at | MGP | Matrix Gla protein | 4.90017E−09 | 3.44 |
| 202672_s_at | ATF3 | Activating transcription factor 3 | 7.34522E−15 | 3.76 |
| 202988_s_at | RGS1 | Regulator of G-protein | 5.04504E−25 | 5.24 |
| | | signaling 1 | | |
| 202995_s_at | FBLN1 | Fibulin 1 | 6.17794E−08 | 2.02 |
| 203821_at | HBEGF | Heparin-binding EGF-like | 4.06679E−13 | 3.31 |
| | | growth factor | | |
| 203851_at | IGFBP6 | Insulin-like growth factor | 2.56813E−12 | 2.00 |
| | | binding protein 6 | | |
| 204622_x_at | NR4A2 | Nuclear receptor subfamily 4, | 1.23506E−10 | 3.38 |
| | | group A, member 2 | | |
| 204955_at | SRPX | Sushi-repeat-containing | 4.15751E−09 | 2.10 |
| | | protein, X-linked | | |
| 205200_at | CLEC3B | C-type lectin domain family | 2.07874E−12 | 2.49 |
| | | 3, member B | | |
| 205239_at | AREG | Amphiregulin | 4.65456E−10 | 2.78 |
| 205382_s_at | CFD | Complement factor D | 4.48062E−10 | 2.18 |
| | | (adipsin) | | |
| 205440_s_at | NPY1R | Neuropeptide Y receptor Y1 | 7.56685E−12 | 2.41 |
| 205799_s_at | SLC3A1 | Solute carrier family 3, | 2.1179E−07 | 2.25 |
| | | member 1 | | |
| 206577_at | VIP | Vasoactive intestinal peptide | 7.96108E−06 | 2.47 |
| 207977_s_at | DPT | Dermatopontin | 7.55307E−12 | 2.04 |
| 208078_s_at | SIK1 | Salt-inducible kinase 1 | 2.81573E−09 | 2.88 |
| 208763_s_at | TSC22D3 | TSC22 domain family, | 1.50914E−14 | 2.33 |
| | | member 3 | | |
| 209101_at | CTGF | Connective tissue growth | 2.93886E−17 | 3.21 |
| | | factor | | |
| 209189_at | FOS | FBJ murine osteosarcoma | 9.30065E−25 | 9.15 |
| | | viral oncogene homolog | | |
| 209335_at | DCN | Decorin | 8.07853E−12 | 2.45 |
| 210472_at | MT1G | Metallothionein 1G | 1.31269E−08 | 2.25 |
| 210764_s_at | CYR61 | Cysteine-rich, angiogenic | 1.7157E−19 | 3.80 |
| | | inducer, 61 | | |
| 211813_x_at | DCN | Decorin | 3.33982E−11 | 2.13 |
| 211896_s_at | DCN | Decorin | 5.66051E−11 | 2.53 |
| 211998_at | H3F3B | H3 histone, family 3B | 2.99953E−23 | 2.20 |
| | | (H3.3B) | | |
| 212099_at | RHOB | Ras homolog gene family, | 9.0115E−14 | 2.19 |
| | | member B | | |
| 212225_at | EIF1 | Eukaryotic translation | 3.70056E−19 | 2.60 |
| | | initiation factor 1 | | |
| 212977_at | CXCR7 | Chemokine (C-X-C motif) | 3.00534E−13 | 2.13 |
| | | receptor 7 | | |
| 213068_at | DPT | Dermatopontin | 1.96475E−11 | 2.81 |
| 213071_at | DPT | Dermatopontin | 1.93102E−10 | 2.04 |
| 214038_at | CCL8 | Chemokine (C-C motif) | 4.23919E−15 | 3.11 |
| | | ligand 8 | | |
| 216248_s_at | NR4A2 | Nuclear receptor subfamily 4, | 2.09133E−10 | 3.49 |
| | | group A, member 2 | | |
| 216834_at | RGS1 | Regulator of G-protein | 8.9595E−25 | 5.14 |
| | | signaling 1 | | |
| 218541_s_at | C8orf4 | Chromosome 8 open reading | 3.6358E−15 | 4.03 |
| | | frame 4 | | |
| 218730_s_at | OGN | Osteoglycin | 5.34811E−09 | 2.40 |
| 219087_at | ASPN | Asporin | 5.37941E−11 | 2.08 |
| 219230 at | TMEM100 | Transmembrane protein 100 | 6.16856E−15 | 2.26 |
| 219295_s_at | PCOLCE2 | Procollagen C-endopeptidase | 8.72205E−10 | 2.41 |
| | | enhancer 2 | | |
| 220037_s_at | LYVE1 | Lymphatic vessel endothelial | 1.23282E−11 | 2.07 |
| | | hyaluronan receptor 1 | | |
| 220468_at | ARL14 | ADP-ribosylation factor-like | 3.09932E−13 | 2.18 |
| | | 14 | | |
| 222162_s_at | ADAMTS1 | ADAM metallopeptidase with | 7.41382E−12 | 2.51 |
| | | thrombospondin type 1 motif, | | |
| | | 1 | | |
| 222722_at | OGN | Osteoglycin | 7.9114E−09 | 2.82 |
| 222943_at | GBA3 | Glucosidase, beta, acid 3 | 6.3056E−07 | 2.33 |
| | | (cytosolic) | | |
| 223121_s_at | SFRP2 | Secreted frizzled-related | 9.25419E−09 | 2.18 |
| | | protein 2 | | |
| 223122_s_at | SFRP2 | Secreted frizzled-related | 5.51482E−10 | 3.32 |
| | | protein 2 | | |
| 224657_at | ERRFI1 | ERBB receptor feedback | 1.2124E−12 | 2.12 |
| | | inhibitor 1 | | |
| 225664_at | COL12A1 | Collagen, type XII, alpha 1 | 7.50679E−10 | 2.03 |
| 225767_at | LOC284801 | Hypothetical protein | 2.83155E−14 | 2.18 |
| | | LOC284801 | | |
| 227099_s_at | C11orf96 | Chromosome 11 open reading | 1.42997E−16 | 3.18 |
| | | frame 96 | | |
| 227404_s_at | EGR1 | Early growth response 1 | 2.56676E−16 | 5.01 |
| 227697_at | SOCS3 | Suppressor of cytokine | 1.39534E−06 | 2.28 |
| | | signaling 3 | | |
| 227827_at | — | — | 0.000197723 | 2.05 |
| 228335_at | CLDN11 | Claudin 11 | 5.1725E−11 | 2.41 |
| 228885_at | MAMDC2 | MAM domain containing 2 | 4.88713E−09 | 2.07 |
| 230494_at | — | — | 8.58621E−15 | 2.65 |
| 243509_at | — | — | 4.91602E−16 | 2.40 |
| 36711_at | MAFF | V-maf musculoaponeurotic | 1.1619E−10 | 2.57 |
| | | fibrosarcoma oncogene | | |
| | | homolog F (avian) | | |
| 38037_at | HBEGF | Heparin-binding EGF-like | 3.03719E−12 | 2.46 |
| | | growth factor | | |
| 1554436_a_at | REG4 | Regenerating islet-derived | 3.97492E−05 | −3.18 |
| | | family, member 4 | | |
| 1558549_s_at | VNN1 | Vanin 1 | 1.46257E−06 | −2.10 |
| 203649_s_at | PLA2G2A | Phospholipase A2, group IIA | 1.56184E−07 | −2.94 |
| | | (platelets, synovial fluid) | | |
| 205242_at | CXCL13 | Chemokine (C-X-C motif) | 0.001696161 | −2.09 |
| | | ligand 13 | | |
| 205844_at | VNN1 | Vanin 1 | 1.64394E−07 | −4.50 |
| 207214_at | SPINK4 | Serine peptidase inhibitor, | 0.00027595 | −2.87 |
| | | Kazal type 4 | | |
| 207397_s_at | HOXD13 | Homeobox D13 | 5.32093E−11 | −3.44 |
| 212531_at | LCN2 | Lipocalin 2 | 7.01758E−09 | −3.24 |
| 212768_s_at | OLFM4 | Olfactomedin 4 | 0.000639115 | −2.79 |
| 214604_at | HOXD11 | Homeobox D11 | 1.29845E−11 | −2.32 |
| 219727_at | DUOX2 | Dual oxidase 2 | 2.23839E−10 | −4.40 |
| 219795_at | SLC6A14 | Solute carrier family 6 (amino | 3.60189E−07 | −4.45 |
| | | acid transporter), member 14 | | |
| 221091_at | INSL5 | Insulin-like 5 | 3.79746E−07 | −3.25 |
| 223447_at | REG4 | Regenerating islet-derived | 2.84257E−05 | −2.78 |
| | | family, member 4 | | |
| 228592_at | MS4A1 | Membrane-spanning 4- | 9.1242E−05 | −2.50 |
| | | domains, subfamily A, | | |
| | | member 1 | | |
| 229152_at | C4orf7 | Chromosome 4 open reading | 0.000120605 | −3.02 |
| | | frame 7 | | |
| 229400_at | HOXD10 | Homeobox D10 | 4.46316E−10 | −3.26 |
| 236681_at | HOXD13 | Homeobox D13 | 2.53117E−11 | −2.30 |
| 238847_at | — | — | 7.09608E−11 | −3.50 |
| 238999_at | — | — | 7.58656E−17 | −2.04 |

TABLE 4
Differentially expressed genes in colon tumor tissue according to a comparison of timepoint. P-values
represent the comparison between tissue obtained presurgery and 10 minutes after resection; the 178
regulated genes with ≧2-fold (statistically significant) change in expression are listed.

| | | | | Proportional |
|---|---|---|---|---|
| Probe ID | Gene | Protein | P-value | change |
| 1555724_s_at | TAGLN | Transgelin | 4.73342E−05 | 2.14 |
| 1555938_x_at | VIM | Vimentin | 6.5247E−06 | 2.06 |
| 1568574_x_at | SPP1 | Secreted phosphoprotein 1 | 0.002506207 | 2.02 |
| 201058_s_at | MYL9 | Myosin, light chain 9, regulatory | 9.1293E−05 | 2.15 |
| 201147_s_at | TIMP3 | TIMP metallopeptidase inhibitor 3 | 1.4351E−05 | 2.15 |
| 201150_s_at | TIMP3 | TIMP metallopeptidase inhibitor 3 | 1.77334E−05 | 2.03 |
| 201289_at | CYR61 | Cysteine-rich, angiogenic | 1.33434E−06 | 2.67 |
| | | inducer, 61 | | |
| 201496_x_at | MYH11 | Myosin, heavy chain 11, smooth | 0.000639767 | 2.02 |
| | | muscle | | |
| 201645_at | TNC | Tenascin C | 0.000236339 | 2.29 |
| 201792_at | AEBP1 | AE binding protein 1 | 6.8465E−05 | 2.03 |
| 202237_at | NNMT | Nicotinamide N- | 2.57535E−05 | 2.15 |
| | | methyltransferase | | |
| 202274_at | ACTG2 | Actin, gamma 2, smooth muscle, | 7.506E−05 | 2.03 |
| | | enteric | | |
| 202291_s_at | MGP | Matrix Gla protein | 1.93448E−05 | 2.77 |
| 202310_s_at | COL1A1 | Collagen, type I, alpha 1 | 0.001917905 | 2.05 |
| 202311_s_at | COL1A1 | Collagen, type I, alpha 1 | 0.002116429 | 2.09 |
| 202363_at | SPOCK1 | Sparc/osteonectin, cwcv and | 3.37525E−05 | 2.19 |
| | | kazal-like domains proteoglycan | | |
| | | (testican) 1 | | |
| 202437_s_at | CYP1B1 | Cytochrome P450, family 1, | 0.001662327 | 2.11 |
| | | subfamily B, polypeptide 1 | | |
| 202498_s_at | SLC2A3 | Solute carrier family 2 | 0.000122207 | 2.00 |
| | | (facilitated glucose transporter), | | |
| | | member 3 | | |
| 202499_s_at | SLC2A3 | Solute carrier family 2 | 0.000238055 | 2.19 |
| | | (facilitated glucose transporter), | | |
| | | member 3 | | |
| 202672_s_at | ATF3 | Activating transcription factor 3 | 2.47058E−05 | 2.02 |
| 202766_s_at | FBN1 | Fibrillin 1 | 5.74137E−05 | 2.08 |
| 202988_s_at | RGS1 | Regulator of G-protein signaling | 1.81321E−09 | 2.97 |
| | | 1 | | |
| 203083_at | THBS2 | Thrombospondin 2 | 5.10405E−05 | 2.70 |
| 204051_s_at | SFRP4 | Secreted frizzled-related protein | 1.8344E−07 | 3.83 |
| | | 4 | | |
| 204052_s_at | SFRP4 | Secreted frizzled-related protein | 2.86372E−07 | 3.32 |
| | | 4 | | |
| 204457_s_at | GAS1 | Growth arrest-specific 1 | 0.000455298 | 2.82 |
| 204472_at | GEM | GTP binding protein | 4.61815E−08 | 2.50 |
| | | overexpressed in skeletal muscle | | |
| 204622_x_at | NR4A2 | Nuclear receptor subfamily 4, | 2.35455E−05 | 2.13 |
| | | group A, member 2 | | |
| 204939_s_at | PLN | Phospholamban | 5.48691E−05 | 2.10 |
| 204940_at | PLN | Phospholamban | 5.82032E−05 | 2.11 |
| 205422_s_at | ITGBL1 | Integrin, beta-like 1 (with EGF- | 1.29825E−05 | 2.91 |
| | | like repeat domains) | | |
| 205547_s_at | TAGLN | Transgelin | 0.000109081 | 2.26 |
| 205713_s_at | COMP | Cartilage oligomeric matrix | 1.01742E−05 | 2.48 |
| | | protein | | |
| 205941_s_at | COL10A1 | Collagen, type X, alpha 1 | 9.47934E−05 | 2.38 |
| 206224_at | CST1 | Cystatin SN | 0.004959398 | 2.06 |
| 206577_at | VIP | Vasoactive intestinal peptide | 0.002985188 | 2.10 |
| 207173_x_at | CDH11 | Cadherin 11, type 2, OB-cadherin | 8.60461E−05 | 2.06 |
| | | (osteoblast) | | |
| 209875_s_at | SPP1 | Secreted phosphoprotein 1 | 0.001536153 | 2.74 |
| 210004_at | OLR1 | Oxidized low density lipoprotein | 0.000130964 | 2.09 |
| | | (lectin-like) receptor 1 | | |
| 210511_s_at | INHBA | Inhibin, beta A | 2.07614E−05 | 2.44 |
| 210517_s_at | AKAP12 | A kinase (PRKA) anchor protein | 1.56563E−05 | 2.04 |
| | | 12 | | |
| 210764_s_at | CYR61 | Cysteine-rich, angiogenic | 2.62979E−06 | 2.40 |
| | | inducer, 61 | | |
| 211122_s_at | CXCL11 | Chemokine (C-X-C motif) ligand | 0.006808687 | 2.10 |
| | | 11 | | |
| 212344_at | SULF1 | sSulfatase 1 | 2.89487E−05 | 2.21 |
| 212353_at | SULF1 | Sulfatase 1 | 9.09842E−06 | 2.64 |
| 212354_at | SULF1 | Sulfatase 1 | 1.30312E−05 | 2.41 |
| 213905_x_at | BGN | Biglycan | 0.000330535 | 2.05 |
| 213943_at | TWIST1 | Twist homolog 1 (Drosophila) | 8.58632E−05 | 2.25 |
| 215033_at | TM4SF1 | Transmembrane 4 L six family | 1.2279E−08 | 2.04 |
| | | member 1 | | |
| 215646_s_at | VCAN | Versican | 0.000677785 | 2.17 |
| 216005_at | TNC | Tenascin C | 4.03919E−05 | 2.39 |
| 216248_s_at | NR4A2 | Nuclear receptor subfamily 4, | 1.26792E−05 | 2.29 |
| | | group A, member 2 | | |
| 216834_at | RGS1 | Regulator of G-protein signaling | 3.05537E−09 | 2.68 |
| | | 1 | | |
| 217428_s_at | COL10A1 | Collagen, type X, alpha 1 | 0.000123852 | 3.64 |
| 218468_s_at | GREM1 | Gremlin 1 | 0.000227583 | 2.72 |
| 218469_at | GREM1 | Gremlin 1 | 0.0003666 | 2.59 |
| 219087_at | ASPN | Asporin | 2.23181E−05 | 2.92 |
| 221730_at | COL5A2 | Collagen, type V, alpha 2 | 0.000692635 | 2.01 |
| 221748_s_at | TNS1 | Tensin 1 | 0.000133411 | 2.03 |
| 222877_at | — | — | 1.4318E−06 | 2.14 |
| 223121_s_at | SFRP2 | Secreted frizzled-related protein 2 | 0.000178028 | 2.59 |
| 223122_s_at | SFRP2 | Secreted frizzled-related protein 2 | 9.3884E−05 | 3.20 |
| 223475_at | CRISPLD1 | Cysteine-rich secretory protein | 1.07185E−05 | 2.17 |
| | | LCCL domain containing 1 | | |
| 224396_s_at | ASPN | Asporin | 6.15766E−06 | 2.54 |
| 224694_at | ANTXR1 | Anthrax toxin receptor 1 | 0.000102969 | 2.25 |
| 225381_at | LOC399959 | Hypothetical LOC399959 | 4.40169E−05 | 2.44 |
| 225481_at | FRMD6 | FERM domain containing 6 | 2.27148E−05 | 2.02 |
| 225664_at | COL12A1 | Collagen, type XII, alpha 1 | 0.000310454 | 2.18 |
| 225681_at | CTHRC1 | Collagen triple helix repeat | 7.13833E−05 | 2.79 |
| | | containing 1 | | |
| 225762_x_at | LOC284801 | Hypothetical protein LOC284801 | 1.00682E−12 | 2.54 |
| 225767_at | LOC284801 | Hypothetical protein LOC284801 | 7.6829E−15 | 4.02 |
| 225782_at | MSRB3 | Methionine sulfoxide reductase | 0.00035065 | 2.01 |
| | | B3 | | |
| 226237_at | COL8A1 | Collagen, type VIII, alpha 1 | 9.79999E−06 | 3.10 |
| 226777_at | ADAM12 | ADAM metallopeptidase domain | 0.00044622 | 2.28 |
| | | 12 | | |
| 226930_at | FNDC1 | Fibronectin type III domain | 2.64629E−06 | 3.01 |
| | | containing 1 | | |
| 227099_s_at | C11orf96 | Chromosome 11 open reading | 1.68701E−06 | 2.48 |
| | | frame 96 | | |
| 227140_at | INHBA | Inhibin, beta A | 4.01738E−05 | 2.97 |
| 227235_at | GUCY1A3 | Guanylate cyclase 1, soluble, | 7.34799E−05 | 2.11 |
| | | alpha 3 | | |
| 227399_at | VGLL3 | Vestigial like 3 (Drosophila) | 0.000940894 | 2.06 |
| 227566_at | NTM | Neurotrimin | 8.58912E−05 | 2.26 |
| 227697_at | SOCS3 | Suppressor of cytokine signaling 3 | 0.000574813 | 2.07 |
| 228202_at | PLN | Phospholamban | 3.4219E−05 | 2.35 |
| 228640_at | PCDH7 | Protocadherin 7 | 0.000418283 | 2.04 |
| 229271_x_at | COL11A1 | Collagen, type XI, alpha 1 | 0.000948907 | 2.06 |
| 229554_at | — | — | 0.000145241 | 2.17 |
| 229802_at | — | — | 0.000922503 | 2.00 |
| 230493_at | SHISA2 | Shisa homolog 2 (Xenopus | 0.000179494 | 2.01 |
| | | laevis) | | |
| 231766_s_at | COL12A1 | Collagen, type XII, alpha 1 | 0.000379392 | 2.28 |
| 231879_at | COL12A1 | Collagen, type XII, alpha 1 | 0.000216652 | 2.29 |
| 232113_at | — | — | 3.02703E−05 | 2.39 |
| 235944_at | HMCN1 | Hemicentin 1 | 7.63204E−06 | 2.17 |
| 236179_at | CDH11 | Cadherin 11, type 2, OB-cadherin | 8.87507E−05 | 2.35 |
| | | (osteoblast) | | |
| 238481_at | MGP | Matrix Gla protein | 1.18316E−06 | 2.53 |
| 238623_at | — | — | 1.04654E−06 | 2.44 |
| 37892_at | COL11A1 | Collagen, type XI, alpha 1 | 0.000101146 | 2.79 |
| 1553828_at | FAM55A | Family with sequence similarity | 0.000633162 | −2.06 |
| | | 55, member A | | |
| 1554436_a_at | REG4 | Regenerating islet-derived | 0.028007727 | −2.52 |
| | | family, member 4 | | |
| 1555962_at | B3GNT7 | UDP-GlcNAc: betaGal beta-1,3- | 0.000795644 | −2.54 |
| | | N-acetylglucosaminyltransferase 7 | | |
| 1555963_x_at | B3GNT7 | UDP-GlcNAc: betaGal beta-1,3- | 0.000789723 | −2.87 |
| | | N-acetylglucosaminyltransferase 7 | | |
| 203240_at | FCGBP | Fc fragment of IgG binding | 0.002948685 | −2.90 |
| | | protein | | |
| 203649_s_at | PLA2G2A | Phospholipase A2, group IIA | 0.003760504 | −2.42 |
| | | (platelets, synovial fluid) | | |
| 203691_at | PI3 | Peptidase inhibitor 3, skin- | 0.002462861 | −2.10 |
| | | derived | | |
| 203908_at | SLC4A4 | Solute carrier family 4, sodium | 0.000983189 | −3.03 |
| | | bicarbonate cotransporter, | | |
| | | member 4 | | |
| 204018_x_at | HBA1 /// | Hemoglobin, alpha 1 /// | 0.000119014 | −2.03 |
| | HBA2 | hemoglobin, alpha 2 | | |
| 204213_at | PIGR | Polymeric immunoglobulin | 0.000398215 | −2.70 |
| | | receptor | | |
| 204673_at | MUC2 | Mucin 2, oligomeric mucus/gel- | 0.005678468 | −2.64 |
| | | forming | | |
| 204818_at | HSD17B2 | Hydroxysteroid (17-beta) | 0.000119211 | −2.26 |
| | | dehydrogenase 2 | | |
| 205097_at | SLC26A2 | Solute carrier family 26 (sulfate | 0.002478216 | −2.39 |
| | | transporter), member 2 | | |
| 205185_at | SPINK5 | Serine peptidase inhibitor, Kazal | 5.58358E−05 | −2.29 |
| | | type 5 | | |
| 205892_s_at | FABP1 | Fatty acid binding protein 1, liver | 0.032075593 | −2.35 |
| 205950_s_at | CA1 | Carbonic anhydrase I | 2.96487E−05 | −3.66 |
| 205979_at | SCGB2A1 | Secretoglobin, family 2A, | 0.000787599 | −2.38 |
| | | member 1 | | |
| 206000_at | MEP1A | Meprin A, alpha (PABA peptide | 0.006828159 | −2.10 |
| | | hydrolase) | | |
| 206143_at | SLC26A3 | Solute carrier family 26, member 3 | 0.002087168 | −4.31 |
| 206198_s_at | CEACAM7 | Carcinoembryonic antigen- | 0.000743488 | −3.77 |
| | | related cell adhesion molecule 7 | | |
| 206199_at | CEACAM7 | Carcinoembryonic antigen- | 0.000617427 | −3.11 |
| | | related cell adhesion molecule 7 | | |
| 206208_at | CA4 | Carbonic anhydrase IV | 0.000142793 | −2.22 |
| 206209_s_at | CA4 | Carbonic anhydrase IV | 5.04159E−05 | −2.98 |
| 206262_at | ADH1C | Alcohol dehydrogenase 1C (class | 5.40013E−05 | −3.66 |
| | | I), gamma polypeptide | | |
| 206268_at | LEFTY1 | Left-right determination factor 1 | 0.018432403 | −2.25 |
| 206561_s_at | AKR1B10 | Aldo-keto reductase family 1, | 0.000169098 | −2.19 |
| | | member B10 (aldose reductase) | | |
| 206641_at | TNFRSF17 | Tumor necrosis factor receptor | 7.48607E−05 | −2.02 |
| | | superfamily, member 17 | | |
| 206664_at | SI | Sucrase-isomaltase (alpha- | 9.10874E−05 | −5.30 |
| | | glucosidase) | | |
| 207003_at | GUCA2A | Guanylate cyclase activator 2A | 2.67886E−05 | −2.88 |
| | | (guanylin) | | |
| 207214_at | SPINK4 | Serine peptidase inhibitor, Kazal | 0.006152697 | −2.96 |
| | | type 4 | | |
| 207245_at | UGT2B17 | UDP glucuronosyltransferase 2 | 0.002178709 | −3.43 |
| | | family, polypeptide B17 | | |
| 208450_at | LGALS2 | Lectin, galactoside-binding, | 1.96728E−05 | −2.58 |
| | | soluble, 2 | | |
| 209116_x_at | HBB | Hemoglobin, beta | 2.82831E−05 | −2.46 |
| 209301_at | CA2 | Carbonic anhydrase II | 0.000491151 | −2.82 |
| 209374_s_at | IGHM | Immunoglobulin heavy constant | 0.002618513 | −2.23 |
| | | mu | | |
| 209458_x_at | HBA1 /// | Hemoglobin, alpha 1 /// | 9.34895E−05 | −2.12 |
| | HBA2 | hemoglobin, alpha 2 | | |
| 209613_s_at | ADH1B | Alcohol dehydrogenase 1B (class | 0.006916787 | −2.22 |
| | | I), beta polypeptide | | |
| 210107_at | CLCA1 | Chloride channel accessory 1 | 0.000416754 | −4.22 |
| 210738_s_at | SLC4A4 | Solute carrier family 4, sodium | 0.000383647 | −2.11 |
| | | bicarbonate cotransporter, | | |
| | | member 4 | | |
| 211696_x_at | HBB | Hemoglobin, beta | 1.18856E−05 | −2.30 |
| 211699_x_at | HBA1 /// | Hemoglobin, alpha 1 /// | 0.000128052 | −2.02 |
| | HBA2 | hemoglobin, alpha 2 | | |
| 211745_x_at | HBA1 /// | Hemoglobin, alpha 1 /// | 8.1156E−05 | −2.16 |
| | HBA2 | hemoglobin, alpha 2 | | |
| 211848_s_at | CEACAM7 | Carcinoembryonic antigen- | 0.000588658 | −3.12 |
| | | related cell adhesion molecule 7 | | |
| 212531_at | LCN2 | Lipocalin 2 | 0.002337585 | −2.20 |
| 212592_at | IGJ | Immunoglobulin J polypeptide, | 0.000180401 | −3.69 |
| | | linker protein for | | |
| | | immunoglobulin alpha and mu | | |
| | | polypeptides | | |
| 212768_s_at | OLFM4 | Olfactomedin 4 | 0.001184695 | −4.23 |
| 214142_at | ZG16 | Zymogen granule protein 16 | 0.000149205 | −4.79 |
| | | homolog (rat) | | |
| 214414_x_at | HBA1 /// | Hemoglobin, alpha 1 /// | 2.72256E−05 | −2.47 |
| | HBA2 | hemoglobin, alpha 2 | | |
| 214433_s_at | SELENBP1 | Selenium binding protein 1 | 0.000985865 | −2.04 |
| 214598_at | CLDN8 | Claudin 8 | 1.78118E−05 | −4.17 |
| 215657_at | SLC26A3 | Solute carrier family 26, member 3 | 0.008000304 | −2.03 |
| 217022_s_at | IGHA1 /// | Immunoglobulin heavy constant | 0.000214466 | −3.22 |
| | IGHA2 | alpha 1 /// immunoglobulin heavy | | |
| | LOC100126583 | /// hypothetical LOC100126583 | | |
| 217109_at | MUC4 | Mucin 4, cell surface associated | 0.000620192 | −2.84 |
| 217110_s_at | MUC4 | Mucin 4, cell surface associated | 0.000989039 | −2.48 |
| 217232_x_at | HBB | Hemoglobin, beta | 2.09662E−05 | −2.04 |
| 217414_x_at | HBA1 /// | Hemoglobin, alpha 1 /// | 5.94249E−05 | −2.17 |
| | HBA2 | hemoglobin, alpha 2 | | |
| 217546_at | MT1M | Metallothionein 1M | 0.002091263 | −2.47 |
| 219727_at | DUOX2 | Dual oxidase 2 | 0.000497377 | −2.84 |
| 219795_at | SLC6A14 | Solute carrier family 6 (amino | 0.010883367 | −2.29 |
| | | acid transporter), member 14 | | |
| 219948_x_at | UGT2A3 | UDP glucuronosyltransferase 2 | 0.001187812 | −3.07 |
| | | family, polypeptide A3 | | |
| 220026_at | CLCA4 | Chloride channel accessory 4 | 4.4668E−05 | −5.04 |
| 220376_at | LRRC19 | Leucine rich repeat containing 19 | 0.001283828 | −2.46 |
| 220834_at | MS4A12 | Membrane-spanning 4-domains, | 1.03895E−05 | −4.65 |
| | | subfamily A, member 12 | | |
| 223447_at | REG4 | Regenerating islet-derived | 0.027839534 | −2.23 |
| | | family, member 4 | | |
| 223597_at | ITLN1 | Intelectin 1 (galactofuranose | 0.000148222 | −4.89 |
| | | binding) | | |
| 224027_at | CCL28 | Chemokine (C-C motif) ligand 28 | 4.55114E−05 | −2.27 |
| 224412_s_at | TRPM6 | Transient receptor potential | 0.000748472 | −2.26 |
| | | cation channel, subfamily M, | | |
| | | member 6 | | |
| 224959_at | SLC26A2 | Solute carrier family 26 (sulfate | 0.004868048 | −2.10 |
| | | transporter), member 2 | | |
| 224963_at | SLC26A2 | Solute carrier family 26 (sulfate | 0.002598016 | −2.08 |
| | | transporter), member 2 | | |
| 226147_s_at | PIGR | Polymeric immunoglobulin | 0.01084721 | −2.54 |
| | | receptor | | |
| 226654_at | MUC12 | Mucin 12, cell surface associated | 0.009193088 | −2.03 |
| 227725_at | ST6GALNAC1 | ST6 (alpha-N-acetyl-neuraminyl- | 0.004737468 | −2.06 |
| | | 2,3-beta-galactosyl-1,3 | | |
| 227735_s_at | C10orf99 | Chromosome 10 open reading | 0.004911848 | −2.04 |
| | | frame 99 | | |
| 227736_at | C10orf99 | Chromosome 10 open reading | 0.00861509 | −2.22 |
| | | frame 99 | | |
| 228232_s_at | VSIG2 | V-set and immunoglobulin | 0.000105191 | −2.57 |
| | | domain containing 2 | | |
| 228241_at | AGR3 | Anterior gradient homolog 3 | 0.023109441 | −2.16 |
| | | (Xenopus laevis) | | |
| 229070_at | C6orf105 | Chromosome 6 open reading | 9.90567E−06 | −3.45 |
| | | frame 105 | | |
| 229254_at | MFSD4 | Major facilitator superfamily | 0.003950209 | −2.16 |
| | | domain containing 4 | | |
| 234632_x_at | — | — | 0.000302489 | −2.79 |
| 238143_at | LOC646627 | Phospholipase inhibitor | 0.001739348 | −2.10 |
| 238750_at | CCL28 | Chemokine (C-C motif) ligand 28 | 7.86416E−05 | −2.43 |
| 239673_at | — | — | 0.000459064 | −2.00 |
| 242601_at | HEPACAM2 | HEPACAM family member 2 | 0.001268202 | −3.43 |

TABLE 5
Differentially expressed genes in colon tumor tissue according to a comparison of timepoint. P-values
represent the comparison between tissue obtained presurgery and 45 minutes after resection; the 332
regulated genes with ≧2-fold (statistically significant) change in expression are listed.

| | | | | Proportional |
|---|---|---|---|---|
| Probe ID | Gene | Protein | P-value | change |
| 1552309_a_at | NEXN | Nexilin (F actin binding protein) | 8.64128E−07 | 2.21 |
| 1554018_at | GPNMB | Glycoprotein (transmembrane) | 5.28765E−06 | 2.18 |
| | | nmb | | |
| 1554741_s_at | FGF7 /// | Fibroblast growth factor 7 /// | 0.000151345 | 2.18 |
| | KGFLP1 | keratinocyte growth factor- | | |
| | | protein 1 | | |
| | /// | /// keratinocyte growth factor-like | | |
| | KGFLP2 | protein 2 | | |
| 1555229_a_at | C1S | Complement component 1, s | 4.3929E−06 | 2.26 |
| | | subcomponent | | |
| 1555724_s_at | TAGLN | Transgelin | 6.24171E−07 | 2.86 |
| 1555778_a_at | POSTN | Periostin, osteoblast specific | 0.002106898 | 2.38 |
| | | factor | | |
| 1556325_at | FILIP1 | Filamin A interacting protein 1 | 8.51026E−06 | 2.11 |
| 1568574_x_at | SPP1 | Secreted phosphoprotein 1 | 1.022E−05 | 2.66 |
| 200986_at | SERPING1 | Serpin peptidase inhibitor, clade | 4.66163E−08 | 2.39 |
| | | G (C1 inhibitor), member 1 | | |
| 201041_s_at | DUSP1 | Dual specificity phosphatase 1 | 1.14772E−05 | 2.21 |
| 201058_s_at | MYL9 | Myosin, light chain 9, regulatory | 2.00947E−06 | 2.92 |
| 201109_s_at | THBS1 | Thrombospondin 1 | 3.33848E−06 | 2.16 |
| 201110_s_at | THBS1 | Thrombospondin 1 | 5.94571E−06 | 2.19 |
| 201141_at | GPNMB | Glycoprotein (transmembrane) | 1.30308E−06 | 2.30 |
| | | nmb | | |
| 201147_s_at | TIMP3 | TIMP metallopeptidase inhibitor 3 | 5.34657E−08 | 2.47 |
| 201150_s_at | TIMP3 | TIMP metallopeptidase inhibitor 3 | 3.1703E−08 | 2.37 |
| 201261_x_at | BGN | Biglycan | 4.85485E−06 | 2.12 |
| 201289_at | CYR61 | Cysteine-rich, angiogenic | 3.96116E−08 | 3.01 |
| | | inducer, 61 | | |
| 201430_s_at | DPYSL3 | Dihydropyrimidinase-like 3 | 4.8262E−09 | 2.46 |
| 201431_s_at | DPYSL3 | Dihydropyrimidinase-like 3 | 1.33119E−08 | 2.41 |
| 201496_x_at | MYH11 | Myosin, heavy chain 11, smooth | 4.17357E−06 | 3.59 |
| | | muscle | | |
| 201497_x_at | MYH11 | Myosin, heavy chain 11, smooth | 6.93199E−06 | 2.78 |
| | | muscle | | |
| 201616_s_at | CALD1 | Caldesmon 1 | 6.51618E−06 | 2.40 |
| 201617_x_at | CALD1 | Caldesmon 1 | 2.0887E−05 | 2.27 |
| 201645_at | TNC | Tenascin C | 5.77047E−06 | 2.80 |
| 201792_at | AEBP1 | AE binding protein 1 | 8.37051E−07 | 2.26 |
| 201842_s_at | EFEMP1 | EGF-containing fibulin-like | 1.49905E−05 | 2.29 |
| | | extracellular matrix protein 1 | | |
| 201843_s_at | EFEMP1 | EGF-containing fibulin-like | 1.47168E−05 | 2.26 |
| | | extracellular matrix protein 1 | | |
| 202133_at | WWTR1 | WW domain containing | 7.44281E−07 | 2.47 |
| | | transcription regulator 1 | | |
| 202207_at | ARL4C | ADP-ribosylation factor-like 4C | 6.6529E−09 | 2.25 |
| 202222_s_at | DES | Desmin | 1.79349E−06 | 3.31 |
| 202237_at | NNMT | Nicotinamide N- | 5.67217E−07 | 2.66 |
| | | methyltransferase | | |
| 202238_s_at | NNMT | Nicotinamide N- | 2.18919E−07 | 2.46 |
| | | methyltransferase | | |
| 202274_at | ACTG2 | Actin, gamma 2, smooth muscle, | 4.68424E−07 | 3.52 |
| | | enteric | | |
| 202291_s_at | MGP | Matrix Gla protein | 6.19879E−07 | 3.62 |
| 202310_s_at | COL1A1 | Collagen, type I, alpha 1 | 0.000563428 | 2.20 |
| 202311_s_at | COL1A1 | Collagen, type I, alpha 1 | 0.000616479 | 2.23 |
| 202363_at | SPOCK1 | Sparc/osteonectin, cwcv and | 4.23499E−08 | 2.97 |
| | | kazal-like domains proteoglycan | | |
| | | (testican) | | |
| 202403_s_at | COL1A2 | Collagen, type I, alpha 2 | 6.92721E−05 | 2.05 |
| 202404_s_at | COL1A2 | Collagen, type I, alpha 2 | 0.000215944 | 2.03 |
| 202437_s_at | CYP1B1 | Cytochrome P450, family 1, | 7.75893E−06 | 3.21 |
| | | subfamily B, polypeptide 1 | | |
| 202498_s_at | SLC2A3 | Solute carrier family 2 | 5.8174E−06 | 2.26 |
| | | (facilitated glucose transporter), | | |
| | | member 3 | | |
| 202499_s_at | SLC2A3 | Solute carrier family 2 | 4.31837E−06 | 2.70 |
| | | (facilitated glucose transporter), | | |
| | | member 3 | | |
| 202555_s_at | MYLK | Myosin light chain kinase | 2.82138E−06 | 2.58 |
| 202628_s_at | SERPINE1 | Serpin peptidase inhibitor, clade | 0.000116651 | 2.03 |
| | | E (nexin, plasminogen activator) | | |
| 202766_s_at | FBN1 | Fibrillin 1 | 1.74402E−06 | 2.35 |
| 202988_s_at | RGS1 | Regulator of G-protein signaling | 7.61767E−12 | 3.77 |
| | | 1 | | |
| 203083_at | THBS2 | Thrombospondin 2 | 1.97222E−06 | 3.01 |
| 203381_s_at | APOE | Apolipoprotein E | 6.47221E−06 | 2.02 |
| 203951_at | CNN1 | Aalponin 1, basic, smooth muscle | 5.34586E−07 | 3.30 |
| 204030_s_at | IQCJ-SCHIP1 | IQ motif containing J- | 3.253E−08 | 2.21 |
| | | schwannomin interacting protein | | |
| | | 1 read- | | |
| | /// | /// schwannomin interacting | | |
| | SCHIP1 | protein 1 | | |
| 204051_s_at | SFRP4 | Secreted frizzled-related protein | 8.23252E−10 | 4.55 |
| | | 4 | | |
| 204052_s_at | SFRP4 | Secreted frizzled-related protein | 9.92564E−10 | 3.99 |
| | | 4 | | |
| 204069_at | MEIS1 | Meis homeobox 1 | 2.8713E−06 | 2.15 |
| 204083_s_at | TPM2 | Tropomyosin 2 (beta) | 1.22293E−06 | 2.71 |
| 204135_at | FILIP1L | Filamin A interacting protein 1- | 1.90475E−06 | 2.01 |
| | | like | | |
| 204457_s_at | GAS1 | Growth arrest-specific 1 | 1.60279E−05 | 3.75 |
| 204472_at | GEM | GTP binding protein | 6.16482E−11 | 2.98 |
| | | overexpressed in skeletal muscle | | |
| 204619_s_at | VCAN | Versican | 1.80097E−05 | 2.21 |
| 204620_s_at | VCAN | Versican | 3.28707E−05 | 2.17 |
| 204622_x_at | NR4A2 | Nuclear receptor subfamily 4, | 4.15508E−05 | 2.11 |
| | | group A, member 2 | | |
| 204894_s_at | AOC3 | Amine oxidase, copper | 4.01865E−05 | 2.10 |
| | | containing 3 (vascular adhesion | | |
| | | protein 1) | | |
| 204938_s_at | PLN | Phospholamban | 4.08198E−06 | 3.30 |
| 204939_s_at | PLN | Phospholamban | 1.19057E−06 | 3.38 |
| 204940_at | PLN | Phospholamban | 2.58764E−06 | 3.49 |
| 205422_s_at | ITGBL1 | Integrin, beta-like 1 (with EGF- | 1.09685E−07 | 3.62 |
| | | like repeat domains) | | |
| 205525_at | CALD1 | Caldesmon 1 | 3.18885E−06 | 2.08 |
| 205547_s_at | TAGLN | Transgelin | 1.20093E−06 | 3.12 |
| 205549_at | PCP4 | Purkinje cell protein 4 | 0.001902231 | 2.27 |
| 205713_s_at | COMP | Cartilage oligomeric matrix | 2.80629E−09 | 2.83 |
| | | protein | | |
| 205767_at | EREG | Epiregulin | 0.015105334 | 2.02 |
| 205880_at | PRKD1 | Protein kinase D1 | 4.49555E−07 | 2.02 |
| 205941_s_at | COL10A1 | Collagen, type X, alpha 1 | 1.88032E−06 | 2.85 |
| 206025_s_at | TNFAIP6 | Tumor necrosis factor, alpha- | 0.001039539 | 2.00 |
| | | induced protein 6 | | |
| 206211_at | SELE | Selectin E | 1.84189E−05 | 2.24 |
| 206224_at | CST1 | Cystatin SN | 0.000510955 | 2.54 |
| 206552_s_at | TAC1 | Tachykinin, precursor 1 | 0.009523889 | 2.15 |
| 206577_at | VIP | Vasoactive intestinal peptide | 0.000487317 | 2.57 |
| 207173_x_at | CDH11 | Cadherin 11, type 2, OB-cadherin | 1.13853E−06 | 2.33 |
| | | (osteoblast) | | |
| 207191_s_at | ISLR | Immunoglobulin superfamily | 5.93722E−08 | 2.17 |
| | | containing leucine-rich repeat | | |
| 208078_s_at | SIK1 | Salt-inducible kinase 1 | 5.33998E−08 | 2.02 |
| 208131_s_at | PTGIS | Prostaglandin I2 (prostacyclin) | 1.09137E−06 | 2.37 |
| | | synthase | | |
| 208747_s_at | C1S | Complement component 1, s | 6.15078E−06 | 2.03 |
| | | subcomponent | | |
| 209101_at | CTGF | Connective tissue growth factor | 1.954E−07 | 2.36 |
| 209209_s_at | FERMT2 | Fermitin family member 2 | 5.19974E−06 | 2.09 |
| 209210_s_at | FERMT2 | Fermitin family member 2 | 5.32763E−06 | 2.24 |
| 209335_at | DCN | Decorin | 0.000271945 | 2.11 |
| 209469_at | GPM6A | Glycoprotein M6A | 1.13583E−05 | 2.76 |
| 209470_s_at | GPM6A | Glycoprotein M6A | 2.60505E−05 | 2.41 |
| 209656_s_at | TMEM47 | Transmembrane protein 47 | 2.33312E−05 | 2.04 |
| 209763_at | CHRDL1 | Chordin-like 1 | 1.87586E−05 | 2.10 |
| 209875_s_at | SPP1 | Secreted phosphoprotein 1 | 1.10445E−05 | 3.86 |
| 210004_at | OLR1 | Oxidized low density lipoprotein | 1.40651E−06 | 2.56 |
| | | (lectin-like) receptor 1 | | |
| 210163_at | CXCL11 | Chemokine (C-X-C motif) ligand | 0.00413012 | 2.22 |
| | | 11 | | |
| 210170_at | PDLIM3 | PDZ and LIM domain 3 | 3.57639E−06 | 2.20 |
| 210299_s_at | FHL1 | Four and a half LIM domains 1 | 0.000496099 | 2.08 |
| 210302_s_at | MAB21L2 | Mab-21-like 2 (C. elegans) | 1.37063E−05 | 2.58 |
| 210495_x_at | FN1 | Fibronectin 1 | 0.000177182 | 2.17 |
| 210511_s_at | INHBA | Inhibin, beta A | 1.15757E−06 | 2.64 |
| 210517_s_at | AKAP12 | A kinase (PRKA) anchor protein | 1.34672E−06 | 2.32 |
| | | 12 | | |
| 210764_s_at | CYR61 | Cysteine-rich, angiogenic | 3.77349E−08 | 2.82 |
| | | inducer, 61 | | |
| 210809_s_at | POSTN | Periostin, osteoblast specific | 0.004350075 | 2.01 |
| | | factor | | |
| 211122_s_at | CXCL11 | Chemokine (C-X-C motif) ligand | 0.002741151 | 2.36 |
| | | 11 | | |
| 211571_s_at | VCAN | Versican | 1.69458E−05 | 2.18 |
| 211597_s_at | HOPX | HOP homeobox | 3.29961E−06 | 2.70 |
| 211719_x_at | FN1 | Fibronectin 1 | 0.000199495 | 2.19 |
| 211896_s_at | DCN | Decorin | 0.000239747 | 2.06 |
| 212077_at | CALD1 | Caldesmon 1 | 1.52964E−05 | 2.03 |
| 212158_at | SDC2 | Syndecan 2 | 3.17792E−06 | 2.02 |
| 212344_at | SULF1 | Sulfatase 1 | 6.40288E−08 | 2.68 |
| 212353_at | SULF1 | Sulfatase 1 | 1.53454E−07 | 3.21 |
| 212354_at | SULF1 | Sulfatase 1 | 4.32709E−08 | 2.98 |
| 212464_s_at | FN1 | Fibronectin 1 | 0.000150308 | 2.29 |
| 212489_at | COL5A1 | Collagen, type V, alpha 1 | 7.30323E−05 | 2.00 |
| 212667_at | SPARC | Secreted protein, acidic, cysteine- | 1.81985E−05 | 2.10 |
| | | rich (osteonectin) | | |
| 212730_at | SYNM | Synemin, intermediate filament | 1.15097E−06 | 3.38 |
| | | protein | | |
| 212764_at | ZEB1 | Zinc finger E-box binding | 2.6995E−06 | 2.12 |
| | | homeobox 1 | | |
| 212992_at | AHNAK2 | AHNAK nucleoprotein 2 | 4.32502E−05 | 2.16 |
| 213068_at | DPT | Dermatopontin | 2.8673E−05 | 2.03 |
| 213125_at | OLFML2B | Olfactomedin-like 2B | 2.52307E−06 | 2.07 |
| 213413_at | STON1 | Stonin 1 | 1.97195E−06 | 2.01 |
| 213746_s_at | FLNA | Filamin A, alpha | 3.12413E−06 | 2.10 |
| 213790_at | ADAM12 | ADAM metallopeptidase domain | 2.35778E−05 | 2.08 |
| | | 12 | | |
| 213905_x_at | BGN | Biglycan | 1.46184E−05 | 2.31 |
| 213943_at | TWIST1 | Twist homolog 1 (Drosophila) | 2.0937E−06 | 2.62 |
| 214027_x_at | DES /// | Desmin /// family with sequence | 3.58011E−06 | 2.08 |
| | FAM48A | similarity 48, member A | | |
| 215446_s_at | LOX | Lysyl oxidase | 0.00013411 | 2.14 |
| 215646_s_at | VCAN | Versican | 4.95198E−05 | 2.43 |
| 216005_at | TNC | Tenascin C | 4.21167E−07 | 3.00 |
| 216248_s_at | NR4A2 | Nuclear receptor subfamily 4, | 3.34829E−05 | 2.25 |
| | | group A, member 2 | | |
| 216442_x_at | FN1 | Fibronectin 1 | 0.000155093 | 2.18 |
| 216834_at | RGS1 | regulator of G-protein signaling 1 | 5.20161E−12 | 3.38 |
| 217428_s_at | COL10A1 | collagen, type X, alpha 1 | 2.1723E−06 | 4.79 |
| 217762_s_at | RAB31 | RAB31, member RAS oncogene | 3.89597E−06 | 2.22 |
| | | family | | |
| 217763_s_at | RAB31 | RAB31, member RAS oncogene | 2.27983E−06 | 2.23 |
| | | family | | |
| 217764_s_at | RAB31 | RAB31, member RAS oncogene | 2.67713E−06 | 2.31 |
| | | family | | |
| 217767_at | C3 | Complement component 3 | 3.48531E−05 | 2.04 |
| 217967_s_at | FAM129A | Family with sequence similarity | 8.2629E−05 | 2.02 |
| | | 129, member A | | |
| 218087_s_at | SORBS1 | Sorbin and SH3 domain | 0.000359092 | 2.17 |
| | | containing 1 | | |
| 218468_s_at | GREM1 | Gremlin 1 | 6.57618E−05 | 2.80 |
| 218469_at | GREM1 | Gremlin 1 | 3.60594E−05 | 2.79 |
| 219087_at | ASPN | Asporin | 8.97706E−08 | 3.78 |
| 220088_at | C5AR1 | Complement component 5a | 1.72055E−05 | 2.16 |
| | | receptor 1 | | |
| 221667_s_at | HSPB8 | Heat shock 22 kDa protein 8 | 2.31999E−06 | 2.55 |
| 221729_at | COL5A2 | Collagen, type V, alpha 2 | 5.86389E−05 | 2.13 |
| 221730_at | COL5A2 | Collagen, type V, alpha 2 | 3.28293E−05 | 2.29 |
| 221731_x_at | VCAN | Versican | 2.21485E−05 | 2.14 |
| 221748_s_at | TNS1 | Tensin 1 | 2.42111E−06 | 2.80 |
| 222088_s_at | SLC2A14 | Solute carrier family 2 | 7.50757E−06 | 2.28 |
| | | (facilitated glucose transporter), | | |
| | | member 14 | | |
| | /// | /// solute carrier family 2 | | |
| | SLC2A3 | (facilitated glucose transporter), | | |
| | | member 3 | | |
| 222108_at | AMIGO2 | Adhesion molecule with Ig-like | 1.40265E−05 | 2.06 |
| | | domain 2 | | |
| 222379_at | KCNE4 | Potassium voltage-gated channel, | 6.92326E−07 | 2.14 |
| | | Isk-related family, member 4 | | |
| 222877_at | — | — | 8.94788E−09 | 2.70 |
| 223121_s_at | SFRP2 | Secreted frizzled-related protein 2 | 5.48205E−06 | 3.14 |
| 223122_s_at | SFRP2 | Secreted frizzled-related protein 2 | 2.4782E−06 | 4.10 |
| 223475_at | CRISPLD1 | Cysteine-rich secretory protein | 5.93303E−08 | 2.39 |
| | | LCCL domain containing 1 | | |
| 224396_s_at | ASPN | Asporin | 4.75282E−08 | 3.04 |
| 224560_at | TIMP2 | TIMP metallopeptidase inhibitor 2 | 1.80999E−05 | 2.08 |
| 224694_at | ANTXR1 | Anthrax toxin receptor 1 | 2.0635E−06 | 2.55 |
| 224823_at | MYLK | Myosin light chain kinase | 2.47351E−05 | 2.31 |
| 225242_s_at | CCDC80 | Coiled-coil domain containing 80 | 9.22593E−06 | 2.15 |
| 225381_at | LOC399959 | Hypothetical LOC399959 | 6.77882E−07 | 2.93 |
| 225481_at | FRMD6 | FERM domain containing 6 | 1.90451E−06 | 2.28 |
| 225664_at | COL12A1 | Collagen, type XII, alpha 1 | 4.09385E−05 | 2.36 |
| 225681_at | CTHRC1 | Collagen triple helix repeat | 1.40753E−06 | 3.41 |
| | | containing 1 | | |
| 225688_s_at | PHLDB2 | Pleckstrin homology-like | 2.743E−05 | 2.11 |
| | | domain, family B, member 2 | | |
| 225710_at | GNB4 | Guanine nucleotide binding | 5.4662E−07 | 2.02 |
| | | protein (G protein), beta | | |
| | | polypeptide 4 | | |
| 225720_at | SYNPO2 | Synaptopodin 2 | 4.58445E−05 | 2.30 |
| 225762_x_at | LOC284801 | Hypothetical protein LOC284801 | 1.10206E−14 | 3.22 |
| 225767_at | LOC284801 | Hypothetical protein LOC284801 | 2.5843E−18 | 5.37 |
| 225782_at | MSRB3 | Methionine sulfoxide reductase | 1.67805E−06 | 2.75 |
| | | B3 | | |
| 225790_at | MSRB3 | Methionine sulfoxide reductase | 1.71053E−06 | 2.32 |
| | | B3 | | |
| 225895_at | SYNPO2 | Synaptopodin 2 | 8.00071E−05 | 2.95 |
| 225946_at | RASSF8 | Ras association (RalGDS/AF-6) | 7.28589E−08 | 2.22 |
| | | domain family (N-terminal) | | |
| | | member 8 | | |
| 226066_at | MITF | Microphthalmia-associated | 6.72249E−07 | 2.18 |
| | | transcription factor | | |
| 226103_at | NEXN | Nexilin (F actin binding protein) | 1.96854E−06 | 2.62 |
| 226237_at | COL8A1 | Collagen, type VIII, alpha 1 | 4.10517E−07 | 3.44 |
| 226303_at | PGM5 | Phosphoglucomutase 5 | 7.85843E−05 | 2.21 |
| 226311_at | ADAMTS2 | ADAM metallopeptidase with | 4.69123E−05 | 2.18 |
| | | thrombospondin type 1 motif, 2 | | |
| 226517_at | BCAT1 | Branched chain amino-acid | 2.52937E−05 | 2.06 |
| | | transaminase 1, cytosolic | | |
| 226545_at | CD109 | CD109 molecule | 2.82547E−05 | 2.16 |
| 226677_at | ZNF521 | Zinc finger protein 521 | 7.80947E−07 | 2.09 |
| 226777_at | ADAM12 | ADAM metallopeptidase domain | 3.81694E−05 | 2.54 |
| | | 12 | | |
| 226834_at | — | — | 6.25067E−05 | 2.03 |
| 226930_at | FNDC1 | Fibronectin type III domain | 1.42194E−08 | 3.73 |
| | | containing 1 | | |
| 227061_at | LOC100506621 | Hypothetical LOC100506621 | 1.38888E−05 | 2.15 |
| 227099_s_at | C11orf96 | Chromosome 11 open reading | 2.4922E−08 | 2.90 |
| | | frame 96 | | |
| 227140_at | INHBA | Inhibin, beta A | 4.82673E−06 | 3.22 |
| 227235_at | GUCY1A3 | Guanylate cyclase 1, soluble, | 9.90574E−07 | 2.68 |
| | | alpha 3 | | |
| 227236_at | TSPAN2 | Tetraspanin 2 | 1.15802E−07 | 2.14 |
| 227399_at | VGLL3 | Vestigial like 3 (Drosophila) | 2.88028E−05 | 2.37 |
| 227529_s_at | AKAP12 | A kinase (PRKA) anchor protein | 1.63519E−06 | 2.38 |
| | | 12 | | |
| 227566_at | NTM | Neurotrimin | 3.275E−07 | 2.71 |
| 227623_at | CACNA2D1 | Calcium channel, voltage- | 1.48825E−06 | 2.29 |
| | | dependent, alpha 2/delta subunit | | |
| | | 1 | | |
| 227662_at | SYNPO2 | Synaptopodin 2 | 0.000101105 | 2.92 |
| 227697_at | SOCS3 | Suppressor of cytokine signaling 3 | 5.55613E−05 | 2.40 |
| 227826_s_at | — | — | 8.06379E−06 | 3.36 |
| 227827_at | — | — | 4.25676E−06 | 3.76 |
| 228133_s_at | MYH11 | Myosin, heavy chain 11, smooth | 2.56294E−05 | 2.60 |
| | | muscle | | |
| 228186_s_at | RSPO3 | R-spondin 3 homolog | 4.02828E−06 | 2.01 |
| | | (Xenopus laevis) | | |
| 228202_at | PLN | Phospholamban | 1.90113E−06 | 3.93 |
| 228640_at | PCDH7 | Protocadherin 7 | 7.0205E−06 | 2.50 |
| 229218_at | COL1A2 | Collagen, type I, alpha 2 | 9.85169E−06 | 2.08 |
| 229271_x_at | COL11A1 | Collagen, type XI, alpha 1 | 1.03103E−05 | 2.32 |
| 229530_at | GUCY1A3 | Guanylate cyclase 1, soluble, | 1.16037E−06 | 2.25 |
| | | alpha 3 | | |
| 229554_at | — | — | 1.15598E−05 | 2.32 |
| 229802_at | — | — | 3.6632E−05 | 2.37 |
| 230493_at | SHISA2 | Shisa homolog 2 | 1.7547E−06 | 2.18 |
| | | (Xenopus laevis) | | |
| 230746_s_at | LOC100288985 | Hypothetical protein | 2.98305E−07 | 2.10 |
| | | LOC100288985 | | |
| 231579_s_at | TIMP2 | TIMP metallopeptidase inhibitor 2 | 2.79382E−05 | 2.07 |
| 231766_s_at | COL12A1 | Collagen, type XII, alpha 1 | 6.96345E−05 | 2.43 |
| 231879_at | COL12A1 | Collagen, type XII, alpha 1 | 5.57201E−05 | 2.38 |
| 232113_at | — | — | 2.88882E−07 | 2.62 |
| 232298_at | LOC401093 | Hypothetical LOC401093 | 1.32384E−05 | 2.12 |
| 235183_at | — | — | 5.11894E−06 | 2.14 |
| 235944_at | HMCN1 | Hemicentin 1 | 2.89194E−07 | 2.50 |
| 236179_at | CDH11 | Cadherin 11, type 2, OB-cadherin | 2.58445E−06 | 2.56 |
| | | (osteoblast) | | |
| 236297_at | — | — | 1.96659E−07 | 2.21 |
| 238481_at | MGP | Matrix Gla protein | 4.22456E−08 | 3.17 |
| 238623_at | — | — | 1.99645E−08 | 2.74 |
| 32128_at | CCL18 | Chemokine (C-C motif) ligand | 8.52833E−05 | 2.53 |
| | | 18 (pulmonary and activation- | | |
| | | regulated) | | |
| 37512_at | HSD17B6 | Hydroxysteroid (17-beta) | 6.80377E−06 | 2.28 |
| | | dehydrogenase 6 homolog | | |
| | | (mouse) | | |
| 37892_at | COL11A1 | Collagen, type XI, alpha 1 | 9.41527E−07 | 3.34 |
| 1552365_at | SCIN | Scinderin | 0.001827943 | −2.11 |
| 1552502_s_at | RHBDL2 | Rhomboid, veinlet-like 2 | 2.55739E−06 | −2.14 |
| | | (Drosophila) | | |
| 1553828_at | FAM55A | Family with sequence similarity | 2.91387E−05 | −2.48 |
| | | 55, member A | | |
| 1554436_a_at | REG4 | Regenerating islet-derived | 0.000212951 | −5.23 |
| | | family, member 4 | | |
| 1555962_at | B3GNT7 | UDP-GlcNAc: betaGal beta-1,3- | 7.154E−05 | −3.04 |
| | | N-acetylglucosaminyltransferase 7 | | |
| 1555963_x_at | B3GNT7 | UDP-GlcNAc: betaGal beta-1,3- | 9.15242E−05 | −3.38 |
| | | N-acetylglucosaminyltransferase 7 | | |
| 203240_at | FCGBP | Fc fragment of IgG binding | 4.78956E−05 | −4.30 |
| | | protein | | |
| 203559_s_at | ABP1 | Amiloride binding protein 1 | 7.27109E−07 | −2.20 |
| | | [amine oxidase (copper- | | |
| | | containing)] | | |
| 203649_s_at | PLA2G2A | Phospholipase A2, group IIA | 0.002158679 | −2.46 |
| | | (platelets, synovial fluid) | | |
| 203691_at | PI3 | Peptidase inhibitor 3, skin- | 2.39096E−05 | −2.54 |
| | | derived | | |
| 203908_at | SLC4A4 | Solute carrier family 4, sodium | 1.09103E−05 | −4.34 |
| | | bicarbonate cotransporter, | | |
| | | member 4 | | |
| 204130_at | HSD11B2 | Hydroxysteroid (11-beta) | 3.28616E−06 | −2.02 |
| | | dehydrogenase 2 | | |
| 204213_at | PIGR | Polymeric immunoglobulin | 3.18471E−05 | −3.53 |
| | | receptor | | |
| 204673_at | MUC2 | Mucin 2, oligomeric mucus/gel- | 0.000154428 | −3.61 |
| | | forming | | |
| 204818_at | HSD17B2 | Hydroxysteroid (17-beta) | 1.61903E−05 | −2.56 |
| | | dehydrogenase 2 | | |
| 204895_x_at | MUC4 | Mucin 4, cell surface associated | 4.95801E−05 | −2.17 |
| 205097_at | SLC26A2 | Solute carrier family 26 (sulfate | 0.007081477 | −2.15 |
| | | transporter), member 2 | | |
| 205185_at | SPINK5 | Serine peptidase inhibitor, Kazal | 1.51319E−05 | −2.36 |
| | | type 5 | | |
| 205259_at | NR3C2 | Nuclear receptor subfamily 3, | 0.000364922 | −2.02 |
| | | group C, member 2 | | |
| 205825_at | PCSK1 | Proprotein convertase | 0.016576148 | −2.16 |
| | | subtilisin/kexin type 1 | | |
| 205950_s_at | CA1 | Carbonic anhydrase I | 1.42429E−06 | −4.31 |
| 205979_at | SCGB2A1 | Secretoglobin, family 2A, | 2.41977E−05 | −3.04 |
| | | member 1 | | |
| 206000_at | MEP1A | Meprin A, alpha (PABA peptide | 0.001001283 | −2.52 |
| | | hydrolase) | | |
| 206143_at | SLC26A3 | Solute carrier family 26, member 3 | 0.00055994 | −4.58 |
| 206198_s_at | CEACAM7 | Carcinoembryonic antigen- | 0.000346086 | −3.98 |
| | | related cell adhesion molecule 7 | | |
| 206199_at | CEACAM7 | Carcinoembryonic antigen- | 0.000404387 | −3.15 |
| | | related cell adhesion molecule 7 | | |
| 206208_at | CA4 | Carbonic anhydrase IV | 3.14437E−06 | −2.60 |
| 206209_s_at | CA4 | Carbonic anhydrase IV | 9.1296E−07 | −3.60 |
| 206262_at | ADH1C | Alcohol dehydrogenase 1C (class | 1.11801E−05 | −4.05 |
| | | I), gamma polypeptide | | |
| 206268_at | LEFTY1 | Left-right determination factor 1 | 0.006935695 | −2.48 |
| 206561_s_at | AKR1B10 | Aldo-keto reductase family 1, | 0.000110476 | −2.30 |
| | | member B10 (aldose reductase) | | |
| 206641_at | TNFRSF17 | Tumor necrosis factor receptor | 6.65093E−05 | −2.02 |
| | | superfamily, member 17 | | |
| 206664_at | SI | Sucrase-isomaltase (alpha- | 2.842E−07 | −8.14 |
| | | glucosidase) | | |
| 207003_at | GUCA2A | Guanylate cyclase activator 2A | 1.76864E−06 | −3.34 |
| | | (guanylin) | | |
| 207214_at | SPINK4 | Serine peptidase inhibitor, Kazal | 2.99566E−05 | −5.21 |
| | | type 4 | | |
| 207222_at | PLA2G10 | Phospholipase A2, group X | 1.45449E−05 | −2.09 |
| 207245_at | UGT2B17 | UDP glucuronosyltransferase 2 | 0.002842665 | −3.42 |
| | | family, polypeptide B17 | | |
| 208450_at | LGALS2 | Lectin, galactoside-binding, | 4.05288E−06 | −2.79 |
| | | soluble, 2 | | |
| 209116_x_at | HBB | Hemoglobin, beta | 2.37634E−05 | −2.60 |
| 209301_at | CA2 | Carbonic anhydrase II | 0.000136418 | −3.05 |
| 209458_x_at | HBA1 /// | Hemoglobin, alpha 1 /// | 0.000257361 | −2.06 |
| | HBA2 | hemoglobin, alpha 2 | | |
| 209613_s_at | ADH1B | Alcohol dehydrogenase 1B (class | 0.016020505 | −2.04 |
| | | I), beta polypeptide | | |
| 209752_at | REG1A | Regenerating islet-derived 1 | 0.042615985 | −2.28 |
| | | alpha | | |
| 210107_at | CLCA1 | Chloride channel accessory 1 | 9.72618E−07 | −7.23 |
| 210174_at | NR5A2 | Nuclear receptor subfamily 5, | 2.06347E−05 | −2.02 |
| | | group A, member 2 | | |
| 210738_s_at | SLC4A4 | Solute carrier family 4, sodium | 7.18965E−05 | −2.30 |
| | | bicarbonate cotransporter, | | |
| | | member 4 | | |
| 211696_x_at | HBB | Hemoglobin, beta | 1.12554E−05 | −2.39 |
| 211699_x_at | HBA1 /// | Hemoglobin, alpha 1 /// | 0.000153371 | −2.02 |
| | HBA2 | hemoglobin, alpha 2 | | |
| 211745_x_at | HBA1 /// | Hemoglobin, alpha 1 /// | 0.000163306 | −2.12 |
| | HBA2 | hemoglobin, alpha 2 | | |
| 211848_s_at | CEACAM7 | Carcinoembryonic antigen- | 0.000349566 | −3.17 |
| | | related cell adhesion molecule 7 | | |
| 212531_at | LCN2 | Lipocalin 2 | 5.05852E−05 | −2.77 |
| 212592_at | IGJ | Immunoglobulin J polypeptide, | 4.97675E−05 | −4.06 |
| | | linker protein for | | |
| | | immunoglobulin alpha and mu | | |
| | | polypeptides | | |
| 212768_s_at | OLFM4 | Olfactomedin 4 | 7.64335E−05 | −6.94 |
| 212814_at | AHCYL2 | Adenosylhomocysteinase-like 2 | 5.00094E−07 | −2.14 |
| 214142_at | ZG16 | Zymogen granule protein 16 | 6.0815E−07 | −7.33 |
| | | homolog (rat) | | |
| 214414_x_at | HBA1 /// | Hemoglobin, alpha 1 /// | 4.54995E−05 | −2.49 |
| | HBA2 | hemoglobin, alpha 2 | | |
| 214433_s_at | SELENBP1 | Selenium binding protein 1 | 4.2296E−05 | −2.33 |
| 214598_at | CLDN8 | Claudin 8 | 1.80976E−06 | −4.81 |
| 215657_at | SLC26A3 | Solute carrier family 26, member 3 | 0.002370526 | −2.26 |
| 217022_s_at | IGHA1 /// | Immunoglobulin heavy constant | 2.43669E−05 | −3.50 |
| | IGHA2 | alpha 1 /// immunoglobulin heavy | | |
| | | constant alpha 2 (A2m marker) | | |
| | LOC100126583 | /// hypothetical LOC100126583 | | |
| 217109_at | MUC4 | Mucin 4, cell surface associated | 1.35267E−05 | −3.86 |
| 217110_s_at | MUC4 | Mucin 4, cell surface associated | 3.51375E−05 | −3.27 |
| 217232_x_at | HBB | Hemoglobin, beta | 2.05507E−05 | −2.09 |
| 217238_s_at | ALDOB | Aldolase B, fructose- | 0.00142396 | −2.04 |
| | | bisphosphate | | |
| 217414_x_at | HBA1 /// | Hemoglobin, alpha 1 /// | 0.000140008 | −2.10 |
| | HBA2 | hemoglobin, alpha 2 | | |
| 217546_at | MT1M | Metallothionein 1M | 0.000408888 | −2.82 |
| 219014_at | PLAC8 | Placenta-specific 8 | 0.001224132 | −2.25 |
| 219727_at | DUOX2 | Dual oxidase 2 | 1.335E−05 | −3.53 |
| 219795_at | SLC6A14 | Solute carrier family 6 (amino | 0.000316091 | −3.36 |
| | | acid transporter), member 14 | | |
| 219948_x_at | UGT2A3 | UDP glucuronosyltransferase 2 | 3.91209E−05 | −4.09 |
| | | family, polypeptide A3 | | |
| 220026_at | CLCA4 | Chloride channel accessory 4 | 6.50973E−07 | −6.86 |
| 220030_at | STYK1 | Serine/threonine/tyrosine kinase | 3.96282E−06 | −2.35 |
| | | 1 | | |
| 220075_s_at | CDHR5 | Cadherin-related family member 5 | 2.10493E−05 | −2.43 |
| 220376_at | LRRC19 | Leucine rich repeat containing 19 | 0.000233493 | −2.74 |
| 220645_at | FAM55D | Family with sequence similarity | 6.0041E−05 | −2.21 |
| | | 55, member D | | |
| 220812_s_at | HHLA2 | HERV-H LTR-associating 2 | 1.4047E−06 | −2.09 |
| 220834_at | MS4A12 | Membrane-spanning 4-domains, | 1.79419E−06 | −5.32 |
| | | subfamily A, member 12 | | |
| 221004_s_at | ITM2C | Integral membrane protein 2C | 2.84249E−05 | −2.08 |
| 223447_at | REG4 | Regenerating islet-derived | 0.000189189 | −4.21 |
| | | family, member 4 | | |
| 223484_at | C15orf48 | Chromosome 15 open reading | 1.34775E−05 | −2.09 |
| | | frame 48 | | |
| 223551_at | PKIB | Protein kinase (cAMP-dependent, | 0.001012167 | −2.33 |
| | | catalytic) inhibitor beta | | |
| 223597_at | ITLN1 | Intelectin 1 (galactofuranose | 1.9742E−07 | −8.23 |
| | | binding) | | |
| 223952_x_at | DHRS9 | Dehydrogenase/reductase (SDR | 0.000600741 | −2.08 |
| | | family) member 9 | | |
| 224009_x_at | DHRS9 | Dehydrogenase/reductase (SDR | 0.000308104 | −2.35 |
| | | family) member 9 | | |
| 224027_at | CCL28 | Chemokine (C-C motif) ligand 28 | 6.04109E−07 | −2.63 |
| 224412_s_at | TRPM6 | Transient receptor potential | 0.000220103 | −2.47 |
| | | cation channel, subfamily M, | | |
| | | member 6 | | |
| 226147_s_at | PIGR | Polymeric immunoglobulin | 0.00063626 | −3.95 |
| | | receptor | | |
| 226654_at | MUC12 | Mucin 12, cell surface associated | 0.001674357 | −2.36 |
| 226974_at | NEDD4L | Neural precursor cell expressed, | 4.62046E−06 | −2.10 |
| | | developmentally down-regulated | | |
| | | 4-like | | |
| 227048_at | LAMA1 | Laminin, alpha 1 | 4.34789E−05 | −2.16 |
| 227725_at | ST6GALNAC1 | ST6 (alpha-N-acetyl-neuraminyl- | 0.000642755 | −2.41 |
| | | 2,3-beta-galactosyl-1,3) | | |
| 227735_s_at | C10orf99 | Chromosome 10 open reading | 0.001153096 | −2.28 |
| | | frame 99 | | |
| 227736_at | C10orf99 | Chromosome 10 open reading | 0.003108923 | −2.40 |
| | | frame 99 | | |
| 228232_s_at | VSIG2 | V-set and immunoglobulin | 4.10444E−06 | −3.08 |
| | | domain containing 2 | | |
| 228241_at | AGR3 | Anterior gradient homolog 3 | 0.00398137 | −2.60 |
| | | (Xenopus laevis) | | |
| 229070_at | C6orf105 | Chromosome 6 open reading | 2.99495E−07 | −3.95 |
| | | frame 105 | | |
| 229254_at | MFSD4 | Major facilitator superfamily | 6.75206E−05 | −2.83 |
| | | domain containing 4 | | |
| 229831_at | CNTN3 | Contactin 3 (plasmacytoma | 0.000235636 | −2.11 |
| | | associated) | | |
| 234632_x_at | — | — | 3.1752E−05 | −3.17 |
| 237530_at | — | — | 4.31807E−05 | −2.03 |
| 238143_at | LOC646627 | Phospholipase inhibitor | 0.000458742 | −2.28 |
| 238750_at | CCL28 | Chemokine (C-C motif) ligand 28 | 1.24326E−06 | −2.94 |
| 238846_at | TNFRSF11A | Tumor necrosis factor receptor | 0.000256856 | −2.14 |
| | | superfamily, member 11a, NFKB | | |
| | | activator | | |
| 239370_at | LOC100505633 | Hypothetical LOC100505633 | 6.16647E−06 | −2.10 |
| 239673_at | — | — | 6.3915E−06 | −2.47 |
| 239994_at | — | — | 1.10317E−05 | −2.10 |
| 240856_at | GPR120 | G protein-coupled receptor 120 | 0.000388776 | −2.03 |
| 241994_at | XDH | Xanthine dehydrogenase | 5.03297E−06 | −2.32 |
| 242601_at | HEPACAM2 | HEPACAM family member 2 | 1.47154E−05 | −5.35 |
| 41469_at | PI3 | Peptidase inhibitor 3, skin- | 1.24208E−05 | −2.46 |
| | | derived | | |

### Example 5—Identification of Potential RNA-Based Tissue Quality Biomarkers

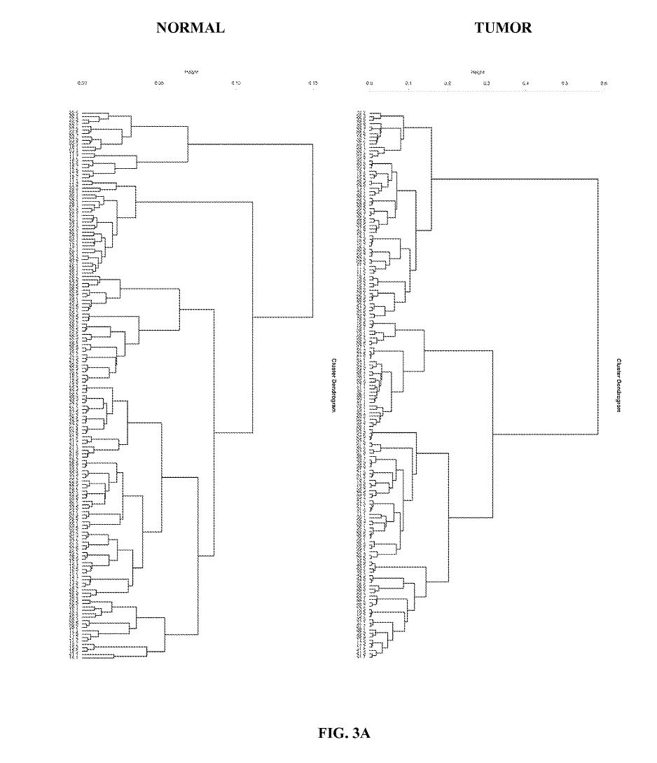

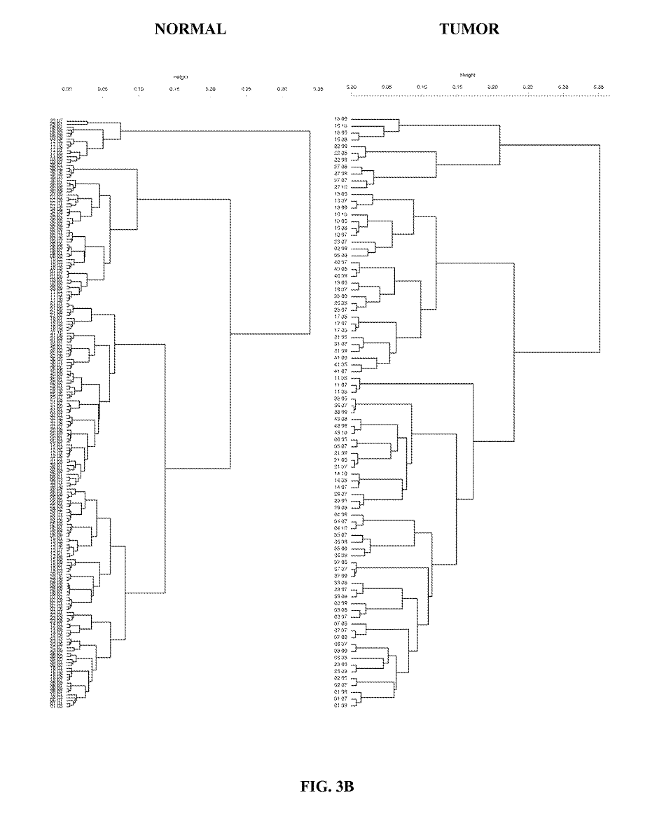

Hierarchical clustering was used to further evaluate gene expression data and to categorize different patient groups. Clustering of data from patients who had colon surgery (normal and tumor tissue clustering separately) resulted in seven different partitions (FIG. 3). Most patients (89% with normal tissue) fell into the partition [presurgery/10′ 20′ 45′] meaning that the presurgery timepoint separated from the 10′, 20′, and 45′ postsurgery timepoints.

Excluding five patients who were regarded as outliers, only patients following the partition presurgery/10′ 20′ 45′ postsurgery in normal tissue were used to compare gene expression intensity levels with multiple t-tests between the presurgery and 10′ postsurgery timepoints, and presurgery and 45′ postsurgery timepoints. In normal colon tissue, 70 probes showed a differential expression in both comparisons. Of these, seven probes (encoding for five different genes) had a log-fold change of ≧2 in both comparisons (see Table 6, below). Expression of another five genes showed ≧2 log-fold change in the comparison presurgery vs. 45′ postsurgery only, while the log-fold change was slightly lower in the presurgery vs. 10′ postsurgery comparison. Genes that were significantly up-regulated upon resection of normal colon tissue comprised transcription factors (EGR1, FOS), signaling molecules (CYR61, RGS1, SGK1) of the extracellular matrix, and dual specificity phosphatase 1 (DUSP1, a protein that dephosphorylates MAPK1). Gene expression for mucosal proteins such as dual oxidase 2 (DUOX2, a protein that plays a role in antimicrobial defense), solute carrier family 6 (SLC6A14, a protein mediating amino acid transport), and vanin 1 (VNN1, a protein involved in vitamin B5 recycling), was down-regulated. In colon tumor tissue, similar gene expression changes were found for CYR61, RGS1, DUSP1, DUOX2, and SLC6A14, although the log-fold change was generally lower compared to normal tissue (see Table 6, below).

The same approach was not applicable to normal liver tissue because of the low number of affected genes and a more diverse change of expression.

TABLE 6
Differentially expressed genes in normal and colorectal tumor tissue. Gene expression was compared: pre, before hepatic
pedicle clamping; post, after clamping; 10′, 10′ after resection; 20′, 20′ after resection; and 45′, 45′ after resection.

| | | | Normal colon tissue | | | | Colorectal tumor tissue | | | |
|---|---|---|---|---|---|---|---|---|---|---|
| | | | Pre vs. 10′ | | Pre vs. 45′ | | Pre vs. 10′ | | Pre vs. 45′ | |
| | | | | Log- | | Log- | | Log- | | Log- |
| Probe | | | p- | fold | p- | fold | p- | fold | p- | fold |
| ID | Gene | Protein and function | value | change | value | change | value | change | value | change |
| 201289_at | CYR61 | Cysteine-rich | 1.57E−18 | 2.49 | 2.84E−22 | 2.55 | 0.00000133 | 1.41 | 3.96E−08 | 1.59 |
| | | angiogenic inducer 61; | | | | | | | | |
| | | extracellular matrix- | | | | | | | | |
| | | associated signaling | | | | | | | | |
| | | protein that plays | | | | | | | | |
| | | important roles in | | | | | | | | |
| | | tissue repair | | | | | | | | |
| 202988_s_at | RGS1 | Regulator of G-protein | 4.21E−21 | 2.34 | 5.05E−25 | 2.39 | 1.81E−09 | 1.57 | 7.62E−12 | 1.91 |
| 216834_at | | signaling 1; attenuates | 1.65E−22 | 2.33 | 8.96E−25 | 2.36 | 3.06E−09 | 1.42 | 5.2E−12 | 1.76 |
| | | signaling activity of | | | | | | | | |
| | | G-proteins | | | | | | | | |
| 201694_s_at | EGR1 | Early growth response | 1.92E−12 | 2.02 | 1.79E−18 | 2.26 | . | . | . | . |
| 227404_s_at | | 1; transcription factor | 1.4E−10 | 1.98 | 2.57E−16 | 2.32 | . | . | . | . |
| 201739_at | SGK1 | Serum/glucocorticoid | 4.03E−14 | 2.09 | 2.74E−16 | 2.21 | . | . | . | . |
| | | regulated kinase 1; | | | | | | | | |
| | | activates potassium, | | | | | | | | |
| | | sodium and chloride | | | | | | | | |
| | | channels | | | | | | | | |
| 209189_at | FOS | FBJ murine | 1.26E−18 | 2.77 | 9.3E−25 | 3.19 | . | . | . | . |
| | | osteosarcoma viral | | | | | | | | |
| | | oncogene homolog; | | | | | | | | |
| | | transcription factor | | | | | | | | |
| | | involved in cell | | | | | | | | |
| | | proliferation, | | | | | | | | |
| | | differentiation, | | | | | | | | |
| | | survival, hypoxia and | | | | | | | | |
| | | angiogenesis | | | | | | | | |
| 218541_s_at | C8orf4 | Chromosome 8 open | 2.39E−12 | 2 | 3.64E−15 | 2.01 | . | . | . | . |
| | | reading frame 4; | | | | | | | | |
| | | uncharacterized | | | | | | | | |
| | | protein | | | | | | | | |
| 201041_s_at | DUSP1 | Dual specificity | 1.21E−17 | 1.85 | 3E−23 | 2.15 | . | . | 0.0000115 | 1.14 |
| | | phosphatase 1; | | | | | | | | |
| | | dephosphorylates | | | | | | | | |
| | | MAP kinase | | | | | | | | |
| | | MAPK1/ERK2 | | | | | | | | |
| 219727_at | DUOX2 | Dual oxidase 2; plays | 3.36E−08 | −1.88 | 2.24E−10 | −2.14 | 0.000497 | −1.51 | 0.0000133 | −1.82 |
| | | a role in antimicrobial | | | | | | | | |
| | | defense at the mucosal | | | | | | | | |
| | | surface | | | | | | | | |
| 219795_at | SLC6A14 | Solute carrier family 6 | 0.0000101 | −1.87 | 0.00000036 | −2.16 | 0.0109 | −1.2 | 0.000316 | −1.75 |
| | | (amino acid | | | | | | | | |
| | | transporter), member | | | | | | | | |
| | | 14; mediates the | | | | | | | | |
| | | uptake of a broad | | | | | | | | |
| | | range of amino acids | | | | | | | | |
| 205844_at | VNN1 | Vanin 1; | 0.000102 | −1.62 | 0.000000164 | −2.17 | . | . | . | . |
| | | amidohydrolase | | | | | | | | |
| | | recycling pantothenic | | | | | | | | |
| | | acid (vitamin B5) and | | | | | | | | |
| | | releasing cysteamine | | | | | | | | |

### Example 6—Identification of Housekeeping Genes not Affected by Surgery and Tissue Processing/Ischemia

Gene expression data were sorted according to the coefficients of variance (CV). The 10 probe sets with the lowest CV across all four timepoints in normal tissue are listed in Table 7 below. While some probe sets encoded for insufficiently explored proteins, among those very constitutively-expressed genes was eukaryotic translation elongation factor 1 alpha 1 (EEF1A1), a widely expressed gene with high copy numbers that is known as a potential housekeeping gene for gene expression analysis (Rienzo et al., 2013). Further well-known housekeeping genes with low CV were ribosomal proteins L13 and S18, beta-glucuronidase, and beta-actin, while other frequently used housekeeping genes, such as beta-2-microglobulin and beta2B-tubulin, were not constitutively expressed.

The same exercise was conducted for gene expression in CRC tissue. Again, EEF1A1 expression showed a very low CV, suggesting it may function as a reference gene in both normal and neoplastic colorectal tissue.

TABLE 7
The 10 probe sets (9 genes) with the lowest coefficient of variation (CV) across all
four timepoints (presurgery, and 10, 20 and 45 minutes after resection) in normal colon
tissue are shown above the dotted line, and 20 well-known housekeeping genes (HKG) with
their CV and their rank when sorted for CV are shown below the dotted line.

| | | | | | Tumor |
|---|---|---|---|---|---|
| | | | Normal tissue | | tissue |
| Probe ID | Gene | Protein and function | CV | Rank | Rank |
| 1558623_at | LOC729121 | Hypothetical LOC729121 | 0.0000651769 | 1 | 22,547 |
| 227472_at | DDA1 | DET1 and DDB1 | 0.0000763301 | 2 | 5,095 |
| | | associated 1; may be | | | |
| | | involved in ubiquitination | | | |
| | | and subsequent | | | |
| | | proteosomal degradation | | | |
| 213477_x_at | EEF1A1 | Eukaryotic translation | 0.000120035 | 3 | 70 |
| 206559_x_at | | elongation factor 1 alpha 1; | 0.00021194 | 10 | 464 |
| | | involved in protein | | | |
| | | biosynthesis | | | |
| 203172_at | FXR2 | Fragile X mental | 0.00012495 | 4 | 5,833 |
| | | retardation, autosomal | | | |
| | | homolog 2; RNA-binding | | | |
| | | protein | | | |
| 202652_at | APBB1 | Amyloid beta (A4) | 0.000140598 | 5 | 22,323 |
| | | precursor protein-binding, | | | |
| | | family B, member 1; | | | |
| | | transcription coregulator | | | |
| 209394_at | ASMTL | Acetylserotonin O- | 0.000153558 | 6 | 28,176 |
| | | methyltransferase-like; | | | |
| | | unknown function | | | |
| 234891_at | DKFZP547L112 | Hypothetical protein | 0.000198356 | 7 | 17,884 |
| | | DKFZp547L112 | | | |
| 212986_s_at | TLK2 | Tousled-like kinase 2; | 0.000208644 | 8 | 2,237 |
| | | involved in chromatin | | | |
| | | assembly | | | |
| 223148_at | PIGS | Phosphatidylinositol | 0.000210952 | 9 | 24,752 |
| | | glycan anchor biosynthesis, | | | |
| | | class S; component of the | | | |
| | | GPI transamidase complex | | | |
| 210646_x_at | RPL13A | Ribosomal protein L13a, | 0.000370481 | 53 | 752 |
| | | HKG | | | |
| 201049_s_at | RPS18 | Ribosomal protein S18, | 0.000581215 | 185 | 2,319 |
| | | HKG | | | |
| 230125_at | GUSB | Glucuronidase, beta, HKG | 0.000939722 | 647 | 19,340 |
| AFFX- | ACTB | Actin, beta, HKG | 0.001080551 | 941 | 7,550 |
| HSAC07/ | | | | | |
| X00351_3_at | | | | | |
| 216457_s_at | SF3A1 | Splicing factor 3a, subunit | 0.001542067 | 2,363 | 890 |
| | | 1, 120 kDa, HKG | | | |
| 212581_x_at | GAPDH | Glyceraldehyde-3- | 0.001735025 | 3,208 | 332 |
| | | phosphate dehydrogenase, | | | |
| | | HKG | | | |
| 201093_x_at | SDHA | Succinate dehydrogenase | 0.002533378 | 8,062 | 42,630 |
| | | complex, subunit A, | | | |
| | | flavoprotein (Fp), HKG | | | |
| 203135_at | TBP | TATA box binding protein, | 0.003036083 | 12,044 | 11,750 |
| | | HKG | | | |
| 203040_s_at | HMBS | Hydroxymethylbilane | 0.003145361 | 12,988 | 6,489 |
| | | synthase, HKG | | | |
| 1565446_at | HPRT1 | Hypoxanthine | 0.003323065 | 14,616 | 23,933 |
| | | phosphoribosyltransferase | | | |
| | | 1, HKG | | | |
| 229165_at | MRPL12 | Mitochondrial ribosomal | 0.003428532 | 15,543 | 12,742 |
| | | protein L12, HKG | | | |
| 224695_at | C2orf29 | Chromosome 2 open | 0.00344027 | 15,651 | 6,081 |
| | | reading frame 29, HKG | | | |
| 211296_x_at | UBC | Ubiquitin C, HKG | 0.003508935 | 16,259 | 2,040 |
| 235741_at | PPIA | Peptidylprolyl isomerase A | 0.003666547 | 17,628 | 33,077 |
| | | (cyclophilin A), HKG | | | |
| 221842_s_at | ZNF131 | Zinc finger protein 131, | 0.003960494 | 20,273 | 20,072 |
| | | HKG | | | |
| 227708_at | EEF1A1 | Eukaryotic translation | 0.004407467 | 24,160 | 24,765 |
| | | elongation factor 1 alpha 1, | | | |
| | | HKG | | | |
| 1566191_at | SUZ12 | Suppressor of zeste 12 | 0.005051028 | 29,227 | 49,826 |
| | | homolog (Drosophila), | | | |
| | | HKG | | | |
| 240686_x_at | TFRC | Transferrin receptor (p90, | 0.006000472 | 35,483 | 30,303 |
| | | CD71), HKG | | | |
| 232311_at | B2M | Beta-2-microglobulin, | 0.012872315 | 49,756 | 44,528 |
| | | HKG | | | |
| 214023_x_at | TUBB2B | Tubulin, beta 2B, HKG | 0.019826709 | 52,506 | 53,778 |

### Example 7—EGFR-Pathway Protein Phosphorylation

EGFR and its downstream key signaling proteins of the AKT and MAPK pathway were investigated in relation to total protein concentration and phosphorylation status. Using expression levels of presurgery biopsies as a reference, changes (up or down) in protein expression of ≧2-fold were documented. In normal liver tissue the preclamping tissue biopsy was used as reference.

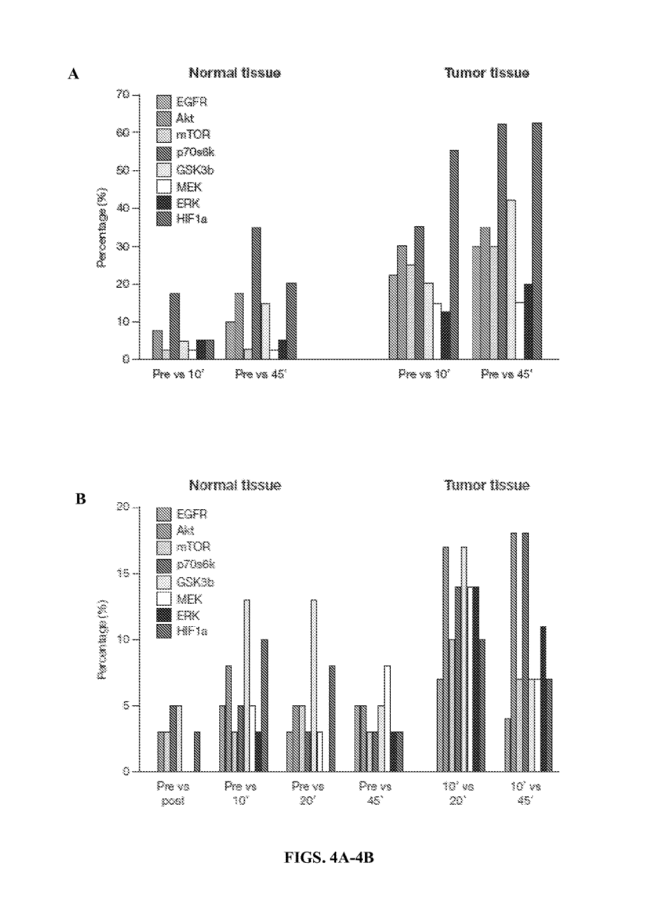

Overall, changes in the phosphorylation of EGFR-pathway proteins were lowest in normal liver, higher in normal colon and highest in cancer tissue (FIGS. 4A-4B). In liver tissue, a change in total protein expression was not observed, while in normal colon tissue EGFR expression changed ≧2-fold in 10% of patients, while expression of the downstream protein p70-S6K changed >2-fold in 35% of patients between presurgery and 45′ postsurgery. In CRC tissue, expression changes in EGFR-related proteins were striking. Between presurgery and 10′ postsurgery, EGFR total protein expression changed by >2-fold in 20% of all patients and in 30% of all patients within a further 35 minutes (presurgery/45′ postsurgery). Subsequently, expression of downstream proteins, such as p70-S6K, changed >2-fold from presurgery to 45′ postsurgery in more than 60% of patients.

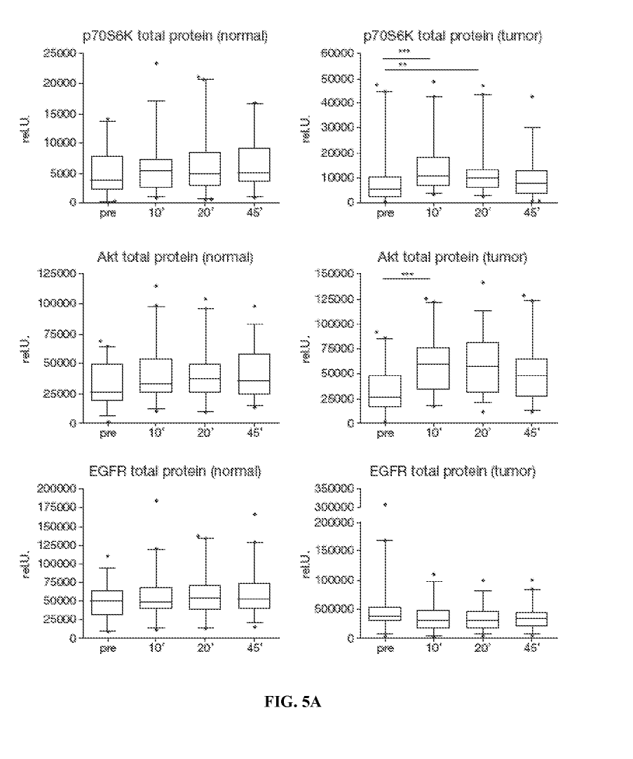

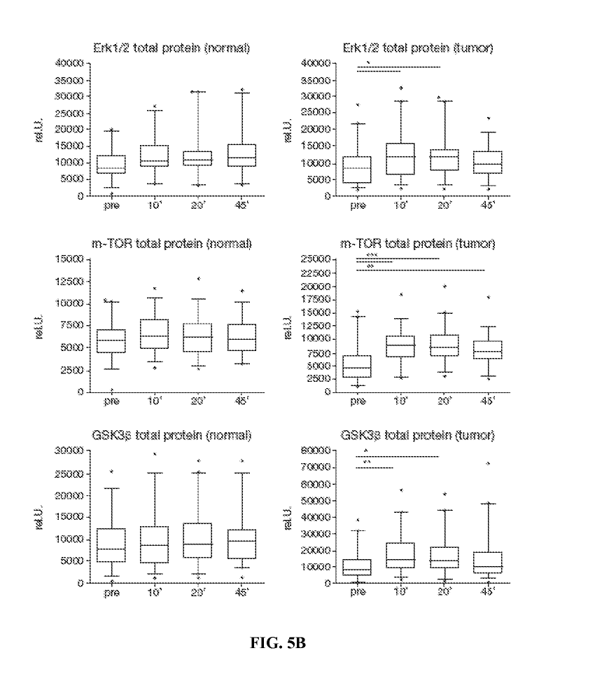

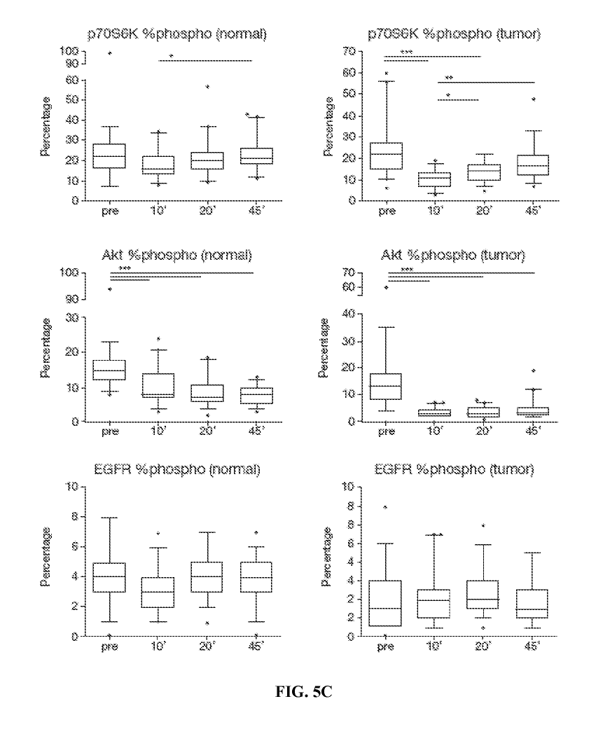

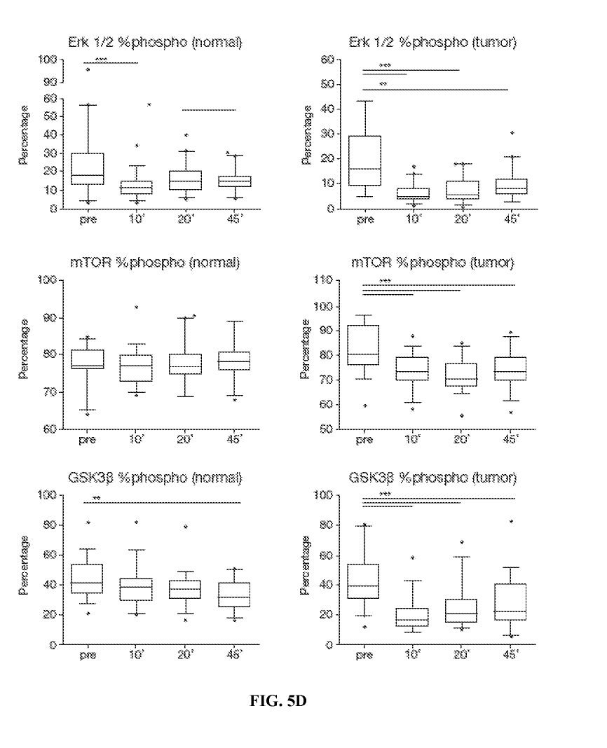

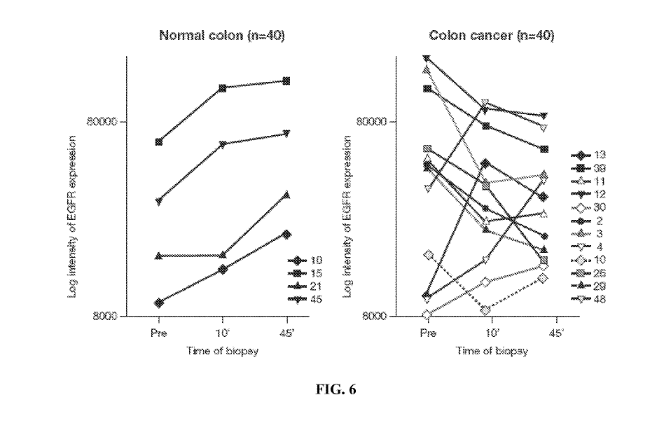

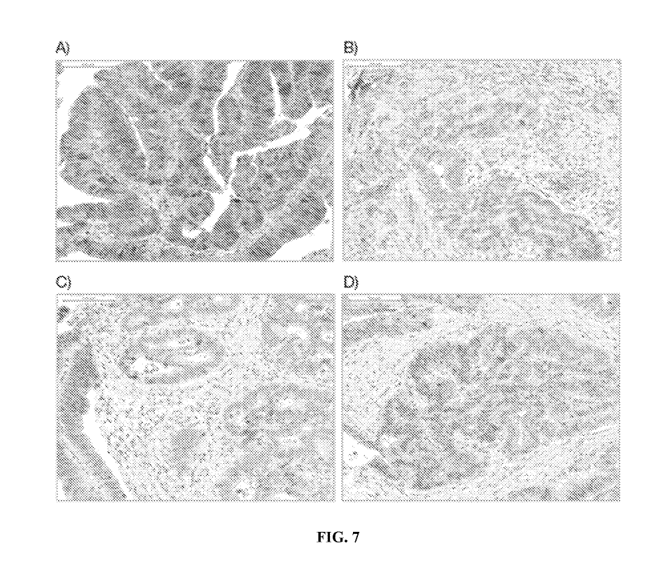

While the change in total protein level became statistically significant for AKT, mTOR, ERK1/2, GSK3B, p70-S6K, HIF1A, and other proteins (FIGS. 5A-5B) such as EGFR showed up- and down-regulation in individual patients and, while still showing unstable expression in some patients, it was not statistically significant (FIG. 6).

### Example 8—the Effect of Tissue Processing Timepoint on Phosphorylation of Key Signaling Molecules within the AKT and MAPK Pathway and HSP27

The phosphorylation status of key signaling proteins was significantly affected in most patients and to a larger extent in tumor tissue compared with normal tissue. The inventors found a chain of phosphorylation events indicating activation in some and inactivation in other parts of the phosphorylation cascade. Statistically significant changes in most key regulatory proteins between presurgery and 10′ postsurgery samples and additional changes for some proteins during the postsurgical cold ischemia time were found. This included AKT, mTOR, ERK1/2, and MEK (FIGS. 5C-5D).

In normal colon and liver tissue, total levels of most proteins did not differ significantly between timepoints. However, there was a statistically significant increase at 10′ postsurgery for AKT and mTOR in CRC tissue. Protein phosphorylation decreased significantly with warm and cold ischemia in normal colon and CRC tumor tissue (FIGS. 5C-5D).

The above mentioned decline in protein phosphorylation was mostly associated with decreased stain intensity upon immunohistochemistry, which was statistically significant for phosphorylated EGFR, AKT, and ERK1/2. Example images of immunohistochemistry for the detection of p-AKT in a patient with colon cancer in relation to ischemia time are shown in FIG. 7.

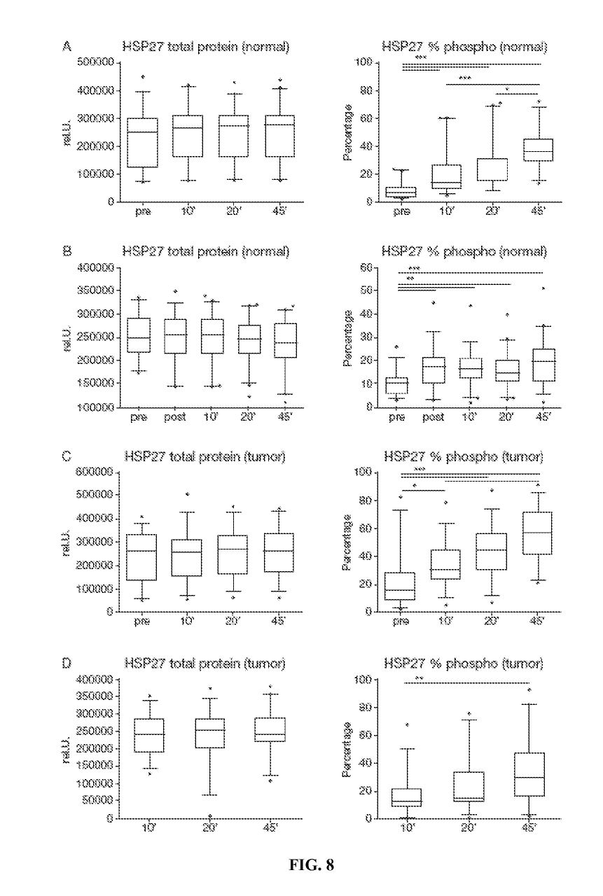

HSP27 was evaluated as its expression is known to respond to cellular stress (Benndorf et al., 1997). While total HSP27 protein levels should be similar across all groups, the percentage of phosphorylated vs. total HSP27 increased over time in an almost linear fashion from presurgery to 45′ postsurgery in most patients, demonstrating a statistically significant difference between presurgery/preclamping samples vs. those taken after prolonged cold ischemia (FIG. 8). After 45′ postsurgery of cold ischemia, the proportion of HSP27 phosphorylation had increased 8-fold compared to presurgery levels. In normal liver tissue it had increased 2-fold.

### Example 9—Statistical Analysis

Statistical analysis of protein content measured on the MSD platform and statistical analysis of staining scores derived from immunohistochemistry was performed with the Kruskal-Wallis test and Dunn's multiple comparisons test, using the software system GraphPad Prism Version 5.0 (GraphPad Software, San Diego, Calif., USA). The significance level was 0.05.

Statistical analysis of changes in gene expression was preceded by normalization using Affymetrix-Power-Tools and log 2 transformation. Symmetry of the data was verified by mean vs. average analysis and boxplots. As a first approach towards statistical data analysis, hierarchical clustering was performed using Spearman correlation coefficient as distance measurement. The ward-linkage method was used to generate dendrograms. Since technical replicates were clustered together in almost all cases, these replicates were averaged and the average was used in further condition clustering, separating samples from normal tissue and those that originated from tumors. Thereafter, separate data across all four timepoints was clustered per individual patient, allowing patients to be grouped into distinct partitions.

Separating normal colon tissue from CRC tissue samples, only patients from the largest partition group were used to compare intensity levels between presurgery and 10′ postsurgery timepoints and presurgery and 45′ postsurgery timepoints. T-tests were performed and p-values were calculated. Probe sets with p>0.001 were excluded as were probe sets with <2 log-fold changes in intensity. The overlap from both comparisons was identified as a list of genes that were particularly vulnerable to warm and cold ischemia. A list of all genes sorted according to the smallest coefficient of variation (CV) was used to identify genes that were apparently not vulnerable to warm and cold ischemia.

For detailed comparison between the three different cold ischemia time intervals, all samples taken at biopsy were excluded from the colorectal data pool. After exclusion, cluster dendrograms across the remaining three timepoints were generated, allowing patients to be grouped into distinct partitions. Patients from the largest partition group were used to compare intensity levels between presurgery and 10′ postsurgery timepoint and presurgery and 45′ postsurgery timepoint. T-tests were performed and p-values were calculated.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bansal et al., R4 RGS proteins: regulation of G-protein signaling and beyond. Pharmacol. Ther., 116:473-495, 2007.
Benndorf and Bielka, Cellular stress response: stress proteins—physiology and implications for cancer. Recent Results Cancer Res., 143:129-144, 1997.
Espina et al., A portrait of tissue phosphoprotein stability in the clinical tissue procurement process. Mol. Cell Proteomics, 7:1998-2018, 2008.
Hatzis et al., Effects of tissue handling on RNA integrity and microarray measurements from resected breast cancers. J. Natl. Cancer Inst., 103:1871-1883, 2011.
Jewell et al., Induction of HIF-la in response to hypoxia is instantaneous. FASEB J., 15:1312-1314, 2001.
Juhl, Preanalytical aspects: a neglected issue. Scand. J. Clin. Lab. Invest. Suppl., 242:63-65, 2010.
Landry et al., Phosphorylation of HSP27 during development and decay of thermotolerance in Chinese hamster cells. J. Cell Physiol., 147:93-101, 1991.
Lau, CCN1/CYR61: the very model of a modern matricellular protein. Cell. Mol. Life Sci., 68:3149-3163, 2011.
Lawan et al., Diversity and specificity of the mitogen-activated protein kinase phosphatase-1 functions. Cell. Mol. Life Sci., 70:223-237, 2013.
Maltseva et al., High-throughput identification of reference genes for research and clinical RT-qPCR analysis of breast cancer samples. J. Clin. Bioinforma., 22:13, 2013.
Rienzo et al., Identification of valid reference housekeeping genes for gene expression analysis in tumor neovascularization studies. Clin. Transl. Oncol., 15:211-218, 2013.
Spruessel et al., Tissue ischemia time affects gene and protein expression patterns within minutes following surgical tumor excision. BioTechniques, 36:1030-1037, 2004.
Tai et al., Hypoxic stress-induced changes in adrenergic function: role of HIF1 alpha. J. Neurochem., 109:513-524, 2009.
Vaught and Lockhart, The evolution of biobanking best practices. Clin. Chim. Acta., 413:1569-1575, 2012.
Zatloukal and Hainaut, Human tissue biobanks as instruments for drug discovery and development: impact on personalized medicine. Biomark. Med., 4:895-903, 2010.

What is claimed is:

1. An in vitro method of determining a quality of a patient sample comprising:
(a) measuring a level of expression of HSP27 protein and a level of phosphorylation of Ser15 of HSP27 protein in the patient sample; and
(b) determining the quality of the patient sample based on the percentage of HSP27 protein phosphorylation in the patient sample.

2. An in vitro method of determining a quality of a patient sample comprising:
(a) measuring a level of expression and a level of phosphorylation of at least two proteins selected from the group consisting of HSP27, EGFR, AKT, mTOR, p70-S6K, GSK3-beta, MEK1/2, and ERK1/2 in the patient sample; and
(b) determining the quality of the patient sample based on the percentage of phosphorylation of said at least two proteins in the patient sample.

3. The method of claim 1 or 2, wherein the sample is a tissue sample.

4. The method of claim 1 or 2, wherein the sample is a frozen sample.

5. The method of claim 1 or 2, wherein the sample is a chemically fixed sample.

6. The method of claim 5, wherein the sample is a formalin-fixed, paraffin-embedded (FFPE) sample.

7. The method of claim 1 or 2, further comprising obtaining a sample from the patient.

8. The method of claim 1 or 2, wherein the sample is obtained from a third party.

9. The method of claim 2, wherein the tissue sample is a tumor resection sample.

10. The method of claim 9, wherein the tumor is a colorectal carcinoma or hepatic carcinoma.

11. The method of claim 1 or 2, wherein a change in the percentage of the protein in the patient sample that is phosphorylated as compared to a reference level indicates the tissue quality.

12. The method of claim 11, wherein the reference level is the level in a biopsy sample.

13. The method of claim 12, wherein the biopsy sample is from the patient.

14. The method of claim 12, wherein the biopsy sample and the patient sample are obtained from the same tissue.

15. The method of claim 1 or 2, wherein determining the quality of the patient sample further comprises estimating the time period that the sample was exposed to cold ischemia conditions.

16. The method of claim 1 or 2, further comprising determining the percentage of at least a second, third or fourth protein in the patient sample that is phosphorylated.

17. The method of claim 16, wherein the second, third or fourth protein is selected from the group consisting of EGFR, AKT, mTOR, p70-S6K, GSK3-beta, MEK1/2, and ERK1/2.

18. The method of claim 16, wherein the percentage of phosphorylation of Tyr1173 of EGFR, Ser 473 of AKT, Ser2448 of mTOR, Thr421 or Ser424 of p70-S6K, Ser9 of GSK3-beta, Ser271/221 of MEK1/2, or Thr202/Tyr204 or Thr185/Tyr187 of ERK1/2 is determined.

19. The method of claim 16, further comprising determining the percentage of 5, 6, 7, 8, 9, or 10 proteins in the patient sample that are phosphorylated.

20. The method of claim 1 or 2, further comprising measuring the level of mRNA expression of at least one gene in the sample selected from the group consisting of CYR61, RGS1, DUSP1, DUOX2, and SLC6A14.

21. The method of claim 20, wherein the level of mRNA expression is measured by quantitative real-time PCR, Northern blotting, in situ hybridization, or an array hybridization.

22. The method of claim 1 or 2, wherein the level of expression of the protein is measured by ELISA, western blotting, mass spectrometry, a capillary immune-detection method or immunohistochemistry.

23. An in vitro method of determining a quality of a patient sample comprising:
(a) measuring a level of expression of at least one mRNA selected from the group consisting of CYR61, RGS1, DUSP1, DUOX2, and SLC6A14 in the patient sample; and
(b) determining the quality of the patient sample based on the level of expression of the at least one mRNA in the patient sample.

24. The method of claim 23, further comprising obtaining a sample from the patient.

25. The method of claim 23, wherein the sample is obtained from a third party.

26. The method of claim 23, further comprising measuring the level of expression of a stable mRNA and determining a ratio of the at least one mRNA to the stable mRNA.

27. The method of claim 26, wherein a change in the ratio as compared to a reference level indicates tissue quality.

28. The method of claim 26, wherein the stable mRNA is EEF1A1.

29. The method of claim 23, further comprising measuring level of expression of 2, 3, 4, 5, 6, 7, 8, 9, or 10 mRNAs in the patient sample.

30. The method of claim 23, further comprising determining the percentage of at least one protein in the patient sample that is phosphorylated.

31. The method of claim 30, wherein the at least one protein is selected from the groups consisting of HSP27, EGFR, AKT, mTOR, p70-S6K, GSK3-beta, MEK1/2, and ERK1/2.

32. The method of claim 31, wherein determining the percentage of at least one protein in the patient sample that is phosphorylated comprises determining the percentage of phosphorylation at Ser15 of HSP27, Tyr1173 of EGFR, Ser 473 of AKT, Ser2448 of mTOR, Thr421 or Ser424 of p70-S6K, Ser9 of GSK3-beta, Ser271/221 of MEK1/2, and/or Thr202/Tyr204 or Thr185/Tyr187 of ERK1/2.

33. The method of claim 31, wherein the at least one protein is HSP27.

34. The method of claim 23, wherein the sample is a tissue sample.

35. The method of claim 23, wherein the sample is a frozen sample.

36. The method of claim 23, wherein the sample is a chemically fixed sample.

37. The method of claim 36, wherein the sample is a formalin-fixed, paraffin-embedded (FFPE) sample.

38. The method of claim 34, wherein the tissue sample is a tumor resection sample.

39. The method of claim 38, wherein the tumor is a colorectal carcinoma or hepatic carcinoma.

40. The method of claim 27, wherein the reference level is the level in a biopsy sample.

41. The method of claim 40, wherein the biopsy sample is from the patient.

42. The method of claim 40, wherein the biopsy sample and the patient sample are obtained from the same tissue.

43. The method of claim 30, wherein the percentage of phosphorylation is determined by ELISA, western blotting, mass spectrometry, a capillary immune-detection method or immunohistochemistry.

44. The method of claim 23, wherein the level of mRNA expression is measured by quantitative real-time PCR, Northern blotting, in situ hybridization, or an array hybridization.

45. An assay for analysis of a sample from a patient comprising selectively measuring a level of expression of HSP27 protein and a level of phosphorylation of Ser15 of HSP27 protein in the patient sample.

46. An assay for analysis of a sample from a patient comprising, selectively measuring a level of expression and a level of phosphorylation of at least two proteins selected from the group consisting of HSP27, EGFR, AKT, mTOR, p70-S6K, GSK3-beta, MEK1/2, and ERK1/2 in the patient sample.

47. The assay of claim 45 or 46, wherein the patient sample is a tissue sample.

48. The assay of claim 45 or 46, wherein the patient sample is a frozen sample.

49. The assay of claim 45 or 46, wherein the sample is a chemically fixed sample.

50. The assay of claim 49, wherein the sample is a formalin-fixed, paraffin-embedded (FFPE) sample.

51. The assay of claim 47, wherein the tissue sample is a tumor resection sample.

52. The assay of claim 47, wherein the tumor is a colorectal carcinoma or hepatic carcinoma.

53. The assay of claim 45 or 46, further comprising obtaining a sample from the patient.

54. The assay of claim 45 or 46, wherein the sample is obtained from a third party.

55. The assay of claim 45 or 46, further comprising selectively measuring a level of expression and a level of phosphorylation of at least two proteins selected from the group consisting of HSP27, EGFR, AKT, mTOR, p70-S6K, GSK3-beta, MEK1/2, and ERK1/2 in a reference sample.

56. The assay of claim 55, wherein the reference sample is a biopsy sample.

57. The assay of claim 56, wherein the biopsy sample is from the patient.

58. The assay of claim 56, wherein the biopsy sample and the patient sample are obtained from the same tissue.

59. The assay of claim 45 or 46, further comprising selectively measuring a level of expression and a level of phosphorylation of at least a second, third or fourth protein.

60. The assay of claim 59, wherein the second, third or fourth protein is selected from the group consisting of HSP27, EGFR, AKT, mTOR, p70-S6K, GSK3-beta, MEK1/2, and ERK1/2.

61. The assay of claim 59, wherein selectively measuring a level of phosphorylation comprises measuring a level of phosphorylation at Ser15 of HSP27, Tyr1173 of EGFR, Ser 473 of AKT, Ser2448 of mTOR, Thr421 or Ser424 of p70-S6K, Ser9 of GSK3-beta, Ser271/221 of MEK1/2, and/or Thr202/Tyr204 or Thr185/Tyr187 of ERK1/2.

62. The assay of claim 60, further comprising selectively measuring a level of expression and a level of phosphorylation of 3, 4, 5, 6, 7, 8, 9, or 10 proteins in the patient sample.

63. The assay of claim 45 or 46, further comprising measuring the level of mRNA expression of at least one gene selected from the group consisting of CYR61, RGS1, DUSP1, DUOX2, and SLC6A14.

64. The assay of claim 63, wherein the level of mRNA expression is measured by quantitative real-time PCR, Northern blotting, in situ hybridization, or an array hybridization.

65. The assay of claim 45 or 46, wherein the level of protein expression of is measured by ELISA, western blotting, mass spectrometry, a capillary immune-detection method or immunohistochemistry.

66. An assay for analysis of a sample from a patient comprising, selectively measuring a level of expression of at least one mRNA selected from the group consisting of CYR61, RGS1, DUSP1, DUOX2, and SLC6A14 in the patient sample.

67. The assay of claim 66, further comprising obtaining a sample from the patient.

68. The assay of claim 66, wherein the sample is obtained from a third party.

69. The assay of claim 66, further comprising selectively measuring the level of expression of a stable mRNA in the sample.

70. The assay of claim 69, wherein the stable mRNA is EEF1A1.

71. The assay of claim 66, further comprising selectively measuring the level of expression of 2, 3, 4, 5, 6, 7, 8, 9, or 10 mRNAs in the patient sample.

72. The assay of claim 66, further comprising measuring a level of expression and a level of phosphorylation of at least a first protein in the patient sample.

73. The assay of claim 66, wherein the first protein is selected from the group consisting of HSP27, EGFR, AKT, mTOR, p70-S6K, GSK3-beta, MEK1/2, and ERK1/2.

74. The assay of claim 73, wherein the first protein is HSP27.

75. The assay of claim 72, wherein measuring a level of expression and a level of phosphorylation of at least a first protein is by ELISA, western blotting, mass spectrometry, a capillary immune-detection method or immunohistochemistry.

76. The assay of claim 66, wherein the sample is a tissue sample.

77. The assay of claim 66, wherein the sample is a frozen sample.

78. The assay of claim 66, wherein the sample is a chemically fixed sample.

79. The assay of claim 78, wherein the sample is a formalin-fixed, paraffin-embedded (FFPE) sample.

80. The assay of claim 76, wherein the tissue sample is a tumor resection sample.

81. The assay of claim 80, wherein the tumor is a colorectal carcinoma or hepatic carcinoma.

82. The assay of claim 66, further comprising selectively measuring a level of expression of at least one mRNA selected from the group consisting of CYR61, RGS1, DUSP1, DUOX2, and SLC6A14 in a reference sample.

83. The assay of claim 82, wherein the reference sample is a biopsy sample.

84. The assay of claim 83, wherein the biopsy sample is from the patient.

85. The assay of claim 83, wherein the biopsy sample and the patient sample are obtained from the same tissue.

86. The assay of claim 66, wherein measuring a level of expression of at least one mRNA is by quantitative real-time PCR, Northern blotting, in situ hybridization, or an array hybridization.
